(19) United States
(12) Patent Application Publication
(10) Pub. No.: US 2026/0350256 A1
(43) Pub. Date: Nov. 5, 2026

(54) MATERIAL MANUFACTURING PROCESS SEARCH METHOD, MATERIAL MANUFACTURING PROCESS SEARCH APPARATUS, AND MATERIAL MANUFACTURING PROCESS SEARCH PROGRAM

(71) Applicant: Resonac Corporation, Tokyo (JP)

(72) Inventor: Takeshi KANESHITA, Tokyo (JP)

(21) Appl. No.: 19/485,022

(22) PCT Filed: Jul. 22, 2024
(86) PCT No.: PCT/JP2024/026134

§ 371 Date: Nov. 17, 2025

(30) Foreign Application Priority Data
Jul. 28, 2023 (JP) 2023-123470

(51) Int. Cl. G06F 30/17; G06Q 50/04

(52) U.S. Cl. CPC G06F 30/17; G06Q 50/04

(57) ABSTRACT

A material manufacturing process search method executed by a computer for designing a design target material includes: analyzing a relationship between a manufacturing process of the design target material and a mechanical property of the design target material; extracting a candidate manufacturing process for the design target material satisfying a mechanical property target value based on an analysis result obtained by analyzing the relationship; calculating data indicating a material structure of the design target material, which is data affecting the mechanical property, by performing thermodynamic calculation for the candidate manufacturing process; and determining appropriateness of the candidate manufacturing process based on the data indicating the material structure calculated by performing the thermodynamic calculation for the candidate manufacturing process.

### TECHNICAL FIELD

The present disclosure relates to a material manufacturing process search method, a material manufacturing process search apparatus, and a material manufacturing process search program.

### BACKGROUND ART

Conventionally, when a material composed of a plurality of compositions or a material manufactured by combining a plurality of manufacturing conditions is designed, trial production is performed while adjusting a “manufacturing process” including a material composition and manufacturing conditions, and “mechanical properties” of a prototype are measured, thereby searching for a manufacturing process for achieving a design target material satisfying desired mechanical properties.

In contrast, for example, if the relationship between a manufacturing process and each mechanical property is analyzed in advance and the analysis results are used to narrow down manufacturing processes for achieving a design target material satisfying desired mechanical properties, an efficient search for a manufacturing process can be performed.

### RELATED-ART DOCUMENTS

### Patent Documents

Patent Document 1: International Publication No. WO2022/264959

### SUMMARY OF THE INVENTION

### Problem to be Solved by the Invention

However, even when the above-described analysis results are used, there may be a case where manufacturing processes cannot be sufficiently narrowed down and many manufacturing processes capable of achieving a design target material satisfying desired mechanical properties are present. In such a case, the workload of trial production and measurement work cannot be reduced, and thus an efficient search for a manufacturing process cannot be performed.

It is an object of the present disclosure to efficiently search for a manufacturing process capable of achieving a design target material satisfying desired mechanical properties.

### Means to Solve the Problem

A first aspect of the present disclosure is a material manufacturing process search method executed by a computer for designing a design target material including a material composed of a plurality of compositions or a material manufactured by combining a plurality of manufacturing conditions, the material manufacturing process search method including:
an analysis step of analyzing a relationship between a manufacturing process of the design target material and a mechanical property of the design target material;
an extraction step of extracting a candidate manufacturing process for the design target material satisfying a mechanical property target value based on an analysis result obtained in the analysis process;
a calculation step of calculating data indicating a material structure of the design target material, which is data affecting the mechanical property, by performing thermodynamic calculation; and
a determination step of determining appropriateness of the candidate manufacturing process based on the data indicating the material structure calculated by performing the thermodynamic calculation for the candidate manufacturing process in the calculation step.

A second aspect of the present disclosure is the material manufacturing process search method according to the first aspect, wherein the design target material is an inorganic material.

A third aspect of the present disclosure is the material manufacturing process search method according to the second aspect, wherein the inorganic material is an alloy material.

A fourth aspect of the present disclosure is the material manufacturing process search method according to the third aspect, wherein the alloy material is a wrought material or a cast material.

A fifth aspect of the present disclosure is the material manufacturing process search method according to the fourth aspect, wherein the wrought material is a wrought aluminum alloy material.

A sixth aspect of the present disclosure is the material manufacturing process search method according to the fifth aspect, wherein
the manufacturing process includes an alloy composition and a manufacturing condition,
the alloy composition includes Si, Mg, Cu, Fe, Zr, and Ti, and
the manufacturing condition includes a homogenization temperature in homogenization treatment, a pre-extrusion heating temperature in solution treatment, a thickness of an extruded cross section, an extrusion pressure, a ram speed, and a cooling water temperature.

A seventh aspect of the present disclosure is the material manufacturing process search method according to the sixth aspect, wherein the mechanical property includes tensile strength, 0.2% proof stress, and elongation.

An eighth aspect of the present disclosure is the material manufacturing process search method according to the seventh aspect, wherein
the data indicating the material structure is data indicating a metallographic structure, and
the data indicating the metallographic structure includes a liquidus temperature, a solid solubility of Mg before extrusion, a solid solubility of Si before extrusion, a precipitate size, a precipitate density, and a volume fraction.

A ninth aspect of the present disclosure is the material manufacturing process search method according to the fourth aspect, wherein the cast material is a cast aluminum alloy material.

A tenth aspect of the present disclosure is the material manufacturing process search method according to the ninth aspect, wherein
the manufacturing process includes an alloy composition and a manufacturing condition,
the alloy composition includes Si, Mg, Cu, Fe, Zr, and Ti, and
the manufacturing condition includes any of a molten metal temperature during pouring, a solution treatment temperature, a solution treatment time, a natural aging time, an artificial aging temperature, an artificial aging time, an annealing temperature, and an annealing time.

An eleventh aspect of the present disclosure is the material manufacturing process search method according to the tenth aspect, wherein the mechanical property includes any of tensile strength, 0.2% proof stress, elongation, Young's modulus, a linear expansion coefficient, and a fatigue property.

A twelfth aspect of the present disclosure is the material manufacturing process search method according to the eleventh aspect, wherein
the data indicating the material structure is data indicating a metallographic structure, and
the data indicating the metallographic structure includes any of a liquidus temperature, solid solubilities of Si, Fe, Cu, Mn, Mg, Cr, Ni, Zn, Ti, V, Pb, Sn, Bi, B, P, Zr, and Sr during solution treatment, a precipitate size, a precipitate density, and a volume fraction.

A thirteenth aspect of the present disclosure is the material manufacturing process search method according to the fourth aspect, wherein the wrought material is a wrought iron alloy material.

A fourteenth aspect of the present disclosure is the material manufacturing process search method according to the thirteenth aspect, wherein
the manufacturing process includes an alloy composition and a manufacturing condition,
the alloy composition includes C, B, N, Si, P, S, Mn, Al, Ti, V, Cr, Co, Ni, Cu, Zr, Nb, Mo, and W, and
the manufacturing condition includes any of a molten metal temperature during iron alloy pouring, a casting speed, an amount of cooling water, an iron alloy heating temperature during hot working, an iron alloy heating time during hot working, a working speed, a rolling reduction, a hot working temperature, a cooling rate after hot working, a natural aging time, a heat treatment temperature, a heat treatment time, and a cooling rate in heat treatment.

A fifteenth aspect of the present disclosure is the material manufacturing process search method according to the fourteenth aspect, wherein the mechanical property includes any of tensile strength, 0.2% proof stress, elongation, Young's modulus, a linear expansion coefficient, an impact property, and a fatigue property.

A sixteenth aspect of the present disclosure is the material manufacturing process search method according to the fifteenth aspect, wherein
the data indicating the material structure is data indicating a metallographic structure, and
the data indicating the metallographic structure includes a liquidus temperature, solid solubilities of C, B, N, Si, P, S, Mn, Al, Ti, V, Cr, Co, Ni, Cu, Zr, Nb, Mo, and W after working, a precipitate size, a precipitate density, and a volume fraction.

A seventeenth aspect of the present disclosure is the material manufacturing process search method according to the fourth aspect, wherein the cast material is a cast iron material.

An eighteenth aspect of the present disclosure is the material manufacturing process search method according to the seventeenth aspect, wherein
the manufacturing process includes an alloy composition and a manufacturing condition, the alloy composition includes C, B, N, Si, P, S, Mn, Al, Ti, V, Cr, Co, Ni, Cu, Zr, Nb, Mo, W, Ca, Mg, and Ce, and
the manufacturing condition includes any of a molten metal temperature during pouring, a pouring speed, a solidification rate, a cooling rate after solidification, a heat treatment temperature, a heat treatment time, and a cooling rate in heat treatment.

A nineteenth aspect of the present disclosure is the material manufacturing process search method according to the eighteenth aspect, herein the mechanical property includes any of tensile strength, 0.2% proof stress, elongation, Young's modulus, a linear expansion coefficient, an impact property, and a fatique property.

A twentieth aspect of the present disclosure is the material manufacturing process search method according to the nineteenth aspect, wherein
the data indicating the material structure is data indicating a metallographic structure, and
the data indicating the metallographic structure includes a liquidus temperature, solid solubilities of C, B, N, Si, P, S, Mn, Al, Ti, V, Cr, Co, Ni, Cu, Zr, Nb, Mo, W, Ca, Mg, and Ce before heat treatment, a precipitate size, a precipitate density, and a volume fraction.

A twenty-first aspect of the present disclosure is the material manufacturing process search method according to the fourth aspect, wherein the wrought material is a wrought copper alloy material.

A twenty-second aspect of the present disclosure is the material manufacturing process search method according to the twenty-first aspect, wherein
the manufacturing process includes an alloy composition and a manufacturing condition,
the alloy composition includes Zn, Pb, Bi, Sn, Fe, P, Al, Hg, Ni, Mn, Se, Te, O, S, Zr, Be, Co, Ti, and As, and
the manufacturing condition includes any of a molten metal temperature during copper alloy pouring, a casting speed, an amount of cooling water, a homogenization temperature, a homogenization time, a cooling rate after homogenization, a copper alloy heating temperature during hot working, a working speed, a cooling rate after working, a natural aging time, an artificial aging temperature, an artificial aging time, a hot working temperature, an annealing temperature, and an annealing time.

A twenty-third aspect of the present disclosure is the material manufacturing process search method according to the twenty-second aspect, wherein the mechanical property includes any of 0.2% proof stress, tensile strength, elongation, electrical conductivity, thermal conductivity, Young's modulus, and a linear expansion coefficient.

A twenty-fourth aspect of the present disclosure is the material manufacturing process search method according to the twenty-third aspect, wherein
the data indicating the material structure is data indicating a metallographic structure, and
the data indicating the metallographic structure includes a liquidus temperature, solid solubilities of Zn, Pb, Bi, Sn, Fe, P, Al, Hg, Ni, Mn, Se, Te, O, S, Zr, Be, Co, Ti, and As before working, a precipitate size, a precipitate density, and a volume fraction.

A twenty-fifth aspect of the present disclosure is the material manufacturing process search method according to the fourth aspect, wherein the cast material is a cast copper alloy material.

A twenty-sixth aspect of the present disclosure is the material manufacturing process search method according to the twenty-fifth aspect, wherein
the manufacturing process includes an alloy composition and a manufacturing condition,
the alloy composition includes Zn, Pb, Bi, Sn, Fe, P, Al, Hg, Ni, Mn, Se, Te, O, S, Zr, Be, Co, Ti, and As, and
the manufacturing condition includes any of a molten metal temperature during pouring, a solution treatment temperature, a solution treatment time, a natural aging time, an artificial aging temperature, an artificial aging time, an annealing temperature, and an annealing time.

A twenty-seventh aspect of the present disclosure is the material manufacturing process search method according to the twenty-sixth aspect, wherein the mechanical property includes any of 0.2% proof stress, tensile strength, elongation, electrical conductivity, thermal conductivity, Young's modulus, and a linear expansion coefficient.

A twenty-eighth aspect of the present disclosure is the material manufacturing process search method according to the twenty-seventh aspect, wherein
the data indicating the material structure is data indicating a metallographic structure, and
the data indicating the metallographic structure includes any of a liquidus temperature, solid solubilities of Zn, Pb, Bi, Sn, Fe, P, Al, Hg, Ni, Mn, Se, Te, O, S, Zr, Be, Co, Ti, and As after solution treatment, a precipitate size, a precipitate density, and a volume fraction.

A twenty-ninth aspect of the present disclosure is the material manufacturing process search method according to the third aspect, wherein the inorganic material is a titanium alloy.

A thirtieth aspect of the present disclosure is the material manufacturing process search method according to the twenty-ninth aspect, wherein
the manufacturing process includes an alloy composition and a manufacturing condition,
the alloy composition includes Al, Sn, V, Mo, Zr, Pd, Si, Cr, Ru, Ta, Co, and Ni, and
the manufacturing condition includes any of a molten metal temperature during pouring, a solution treatment temperature, a solution treatment time, an artificial aging temperature, an artificial aging time, an annealing temperature, and an annealing time.

A thirty-first aspect of the present disclosure is the material manufacturing process search method according to the thirtieth aspect, wherein the mechanical property includes any of 0.2% proof stress, tensile strength, elongation, Young's modulus, a linear expansion coefficient, and a fatigue property.

A thirty-second aspect of the present disclosure is the material manufacturing process search method according to the thirty-first aspect, wherein
the data indicating the material structure is data indicating a metallographic structure, and
the data indicating the metallographic structure includes any of a liquidus temperature, solid solubilities of Al, Sn, V, Mo, Zr, Pd, Si, Cr, Ru, Ta, Co, and Ni during solution treatment, a precipitate size, a precipitate density, and a volume fraction.

A thirty-third aspect is a material manufacturing process search apparatus for designing a design target material including a material composed of a plurality of compositions or a material manufactured by combining a plurality of manufacturing conditions, the material manufacturing process search apparatus including:
an analysis unit configured to analyze a relationship between a manufacturing process of the design target material and a mechanical property of the design target material;
an extraction unit configured to extract a candidate manufacturing process for the design target material satisfying a mechanical property target value based on an analysis result obtained by the analysis unit;
a calculation unit configured to calculate data indicating a material structure of the design target material, which is data affecting the mechanical property, by performing thermodynamic calculation; and
a determination unit configured to determine appropriateness of the candidate manufacturing process based on the data indicating the material structure calculated by the calculation unit performing the thermodynamic calculation for the candidate manufacturing process.

A thirty-fourth aspect is a manufacturing process search program for causing a computer of a material manufacturing process search apparatus for designing a design target material including a material composed of a plurality of compositions or a material manufactured by combining a plurality of manufacturing conditions to execute a process including:
an analysis step of analyzing a relationship between a manufacturing process of the design target material and a mechanical property of the design target material;
an extraction step of extracting a candidate manufacturing process for the design target material satisfying a mechanical property target value based on an analysis result obtained in the analysis process;
a calculation step of calculating data indicating a material structure of the design target material, which is data affecting the mechanical property, by performing thermodynamic calculation; and
a determination step of determining appropriateness of the candidate manufacturing process based on the data indicating the material structure calculated by performing the thermodynamic calculation for the candidate manufacturing process in the calculation step.

### Effects of the Invention

According to the present disclosure, it is possible to efficiently search for a manufacturing process capable of achieving a design target material satisfying desired mechanical properties.

### BRIEF DESCRIPTION OF THE DRAWINGS

FIG. 1 is a diagram illustrating an example of a manufacturing flow of an aluminum alloy extruded profile;

FIG. 2 is a diagram illustrating the relationship between a manufacturing process and each of mechanical properties in the manufacturing flow of the aluminum alloy extruded profile.

FIG. 3 is a diagram illustrating the relationship between the manufacturing process and a metallographic structure in the manufacturing flow of the aluminum alloy extruded profile;

FIG. 4 is a diagram illustrating an outline of processing performed by a manufacturing process search apparatus;

FIG. 5 is a diagram illustrating an example of a hardware configuration of the manufacturing process search apparatus;

FIG. 6 is a diagram illustrating an example of a functional configuration of the manufacturing process search apparatus;

FIG. 7 is a first diagram illustrating a specific example of processing performed by an analysis unit;

FIG. 8 is a diagram illustrating a verification example of analysis results of multiple regression analysis;

FIG. 9 is a diagram illustrating a specific example of processing performed by an extraction unit;

FIG. 10 is a first diagram illustrating a specific example of processing performed by a calculation unit;

FIG. 11 is a diagram illustrating a specific example of processing performed by a determination unit;

FIG. 12 is a flowchart illustrating a flow of manufacturing process search processing;

FIG. 13 is a diagram illustrating an example of search results obtained by manufacturing process search processing;

FIG. 14 is a second diagram illustrating a specific example of processing performed by the analysis unit;

FIG. 15 is a second diagram illustrating a specific example of processing performed by the calculation unit;

FIG. 16 is a third diagram illustrating a specific example of processing performed by the analysis unit;

FIG. 17 is a third diagram illustrating a specific example of processing performed by the calculation unit;

FIG. 18 is a fourth diagram illustrating a specific example of processing performed by the analysis unit;

FIG. 19 is a fourth diagram illustrating a specific example of processing performed by the calculation unit;

FIG. 20 is a fifth diagram illustrating a specific example of processing performed by the analysis unit;

FIG. 21 is a fifth diagram illustrating a specific example of processing performed by the calculation unit;

FIG. 22 is a sixth diagram illustrating a specific example of processing performed by the analysis unit;

FIG. 23 is a sixth diagram illustrating a specific example of processing performed by the calculation unit;

FIG. 24 is a seventh diagram illustrating a specific example of processing performed by the analysis unit; and

FIG. 25 is a seventh diagram illustrating a specific example of processing performed by the calculation unit.

### MODE FOR CARRYING OUT THE INVENTION

Hereinafter, each embodiment will be described with reference to the accompanying drawings. In this specification and the drawings, components having substantially the same functional configurations are denoted by the same reference numerals, and redundant descriptions thereof will be omitted.

### First Embodiment

In a first embodiment, manufacturing process search processing when a design target material is an inorganic material will be described. More specifically, in the first embodiment, manufacturing process search processing when a wrought aluminum alloy material is used as a design target material, from among alloy materials, which are examples of inorganic materials, will be described.

### <Manufacturing Flow of Aluminum Alloy Extruded Profile>

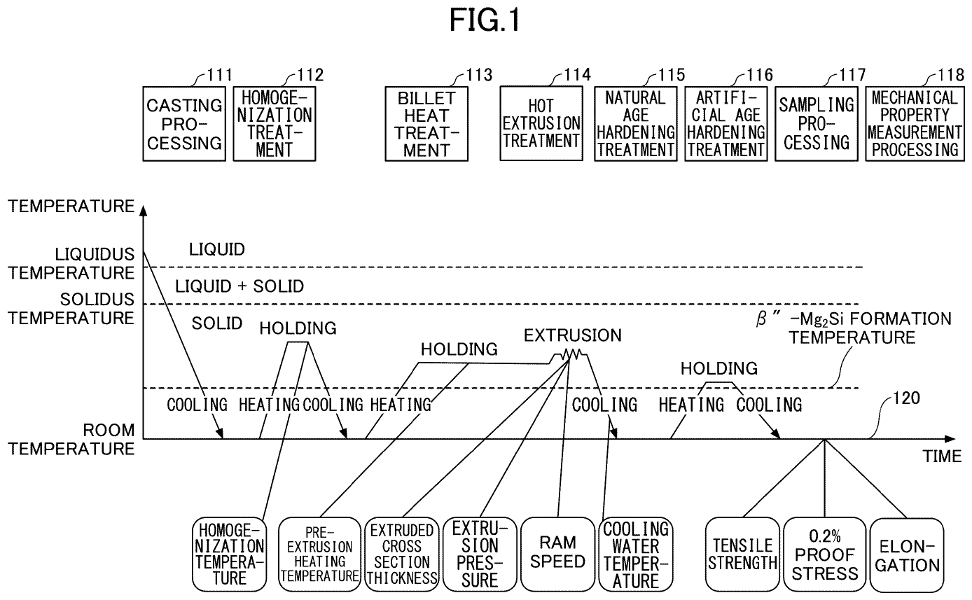

First, as a manufacturing flow of a wrought aluminum alloy material, to which a manufacturing process searched by a material manufacturing process search apparatus (hereinafter simply referred to as a “manufacturing process search apparatus”) according to the first embodiment is applied, a manufacturing flow of an aluminum alloy extruded profile will be described. FIG. 1 is a diagram illustrating an example of the manufacturing flow of the aluminum alloy extruded profile.

As illustrated in FIG. 1, the manufacturing flow of the aluminum alloy extruded profile includes casting treatment 111, homogenization treatment 112, billet heat treatment 113, hot extrusion treatment 114, natural age hardening treatment 115, artificial age hardening treatment 116, sampling processing 117, and mechanical property measurement processing 118. Further, the temperature transition of each processing/treatment included in the manufacturing flow of the aluminum alloy extruded profile is as indicated by reference numeral 120.

The casting treatment 111 refers to treatment in which an aluminum base metal is melted at a liquidus temperature or higher and then additive metals are blended and adjusted to form a cylindrical billet using a casting machine.

The homogenization treatment 112 refers to treatment in which the billet produced in the casting treatment 111 is heated and held at a solidus temperature or lower and then slowly cooled. The structure of the billet produced in the casting treatment 111 includes an uneven distribution of solute atoms, non-equilibrium compound phases, strains caused during cooling, and the like, and the homogenization treatment 112 is performed for the purpose of eliminating them. In the first embodiment, a temperature at the time of heating in the homogenization treatment 112 is referred to as a “homogenization temperature”. Note that, in the first embodiment, the “homogenization temperature” is used as one element of manufacturing conditions included in a manufacturing process.

The billet heat treatment 113 refers to treatment in which the billet subjected to the homogenization treatment 112 is heated and then cut into a predetermined length.

The hot extrusion treatment 114 refers to treatment in which an aluminum alloy extruded profile is formed and treatment in which the formed aluminum alloy extruded profile is quenched using cooling water. The aluminum alloy extruded profile is formed by setting the billet, which is cut in the billet heat treatment 113, in an extruder and then extruding the billet from a die at a high pressure.

In the first embodiment, the temperature of the cut billet before the cut billet is extruded is referred to as a “pre-extrusion heating temperature”. In the first embodiment, the “pre-extrusion heating temperature” is used as one element of manufacturing conditions included in a manufacturing process.

In the first embodiment, the thickness of an extruded cross section is referred to as an “extruded cross section thickness”, and a pressure at which the extruder extrudes the billet from the die is referred to as an “extrusion pressure”. In the first embodiment, each of the “extruded cross section thickness” and the “extrusion pressure” is used as one element of manufacturing conditions included in a manufacturing process.

Further, in the first embodiment, the speed of a ram (push bar) at which the extruder extrudes the cut billet is referred to as a “ram speed”. In the first embodiment, the “ram speed” is used as one element of manufacturing conditions included in a manufacturing process.

In the first embodiment, the temperature of cooling water used to quench the aluminum alloy extruded profile is referred to as a “cooling water temperature”. In the first embodiment, the “cooling water temperature” is used as one element of manufacturing conditions included in a manufacturing process.

The natural age hardening treatment 115 refers to treatment in which elements dissolved in supersaturation are precipitated over time by keeping the aluminum alloy extruded profile, quenched in the hot extrusion treatment 114, at a normal temperature. The natural age hardening treatment 115 allows the aluminum alloy extruded profile to be hardened by precipitating the elements dissolved in supersaturation.

The artificial age hardening treatment 116 refers to treatment in which elements dissolved in supersaturation are artificially precipitated by heating the aluminum alloy extruded profile, subjected to the natural age hardening treatment 115, again at a β″-Mg₂Si formation temperature or higher. The artificial age hardening treatment 116 allows the aluminum alloy extruded profile to be further hardened by artificially precipitating the elements dissolved in supersaturation.

The sampling processing 117 refers to processing in which a portion of the aluminum alloy extruded profile subjected to the artificial age hardening treatment 116 is cut out to measure mechanical properties.

The mechanical property measurement processing 118 refers to processing in which mechanical properties of a portion of the aluminum alloy extruded profile cut out in the sampling processing 117 are measured. In the first embodiment, “tensile strength”, “0.2% proof stress”, and “elongation” are measured as the mechanical properties in the mechanical property measurement processing 118.

<Relationship between Manufacturing Process and Mechanical Properties in Manufacturing Flow of Aluminum Alloy Extruded Profile>

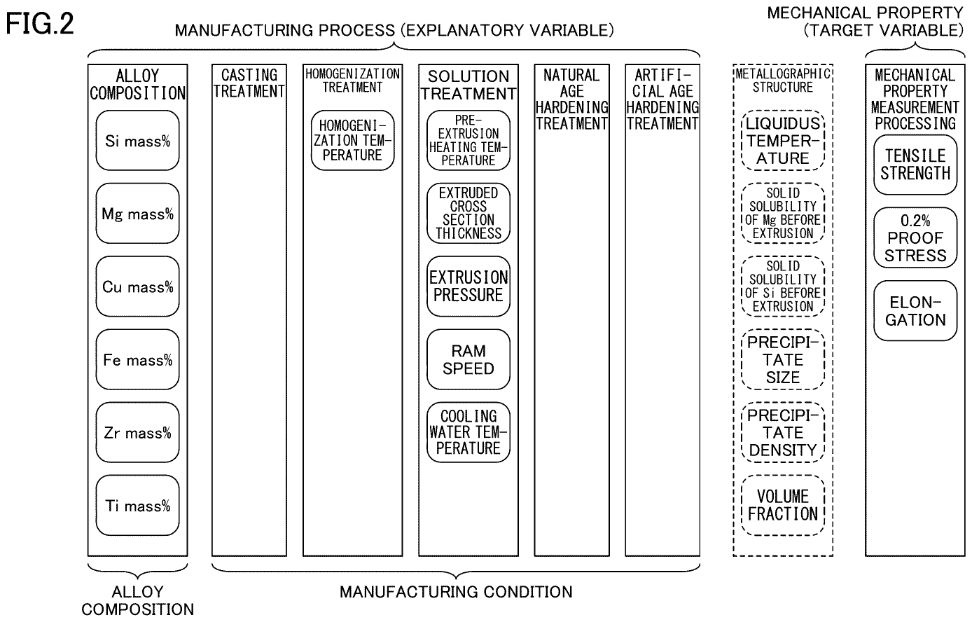

Next, the relationship between a manufacturing process and mechanical properties in the manufacturing flow of the aluminum alloy extruded profile will be described. FIG. 2 is a diagram illustrating the relationship between the manufacturing process and the mechanical properties in the manufacturing flow of the aluminum alloy extruded profile.

As described above, the mechanical properties of the aluminum alloy extruded profile manufactured according to the manufacturing flow are measured in the mechanical property measurement processing 118. The tensile strength, 0.2% proof stress, and elongation, which are the mechanical properties measured at this time, correlate to the manufacturing process including:
an alloy composition of the aluminum alloy extruded profile; and
manufacturing conditions of the aluminum alloy extruded profile.

Therefore, the manufacturing process search apparatus according to the first embodiment analyzes the relationship between the manufacturing process and each of the mechanical properties, by using the alloy composition and the manufacturing conditions of the manufacturing process as “explanatory variables” and each of the mechanical properties as a “target variable”. Note that as illustrated in FIG. 2, in the first embodiment, Si mass %, Mg mass %, Cu mass %, Fe mass %, Zr mass %, and Ti mass % are used as the alloy composition of the aluminum alloy extruded profile.

Further, in the first embodiment, as the manufacturing conditions of the aluminum alloy extruded profile, “homogenization temperature”, “pre-extrusion heating temperature”, “extruded cross section thickness”, “ram speed”, and “cooling water temperature” are used. In FIG. 2, solution treatment in which the “pre-extrusion heating temperature”, the “extruded cross section thickness”, the “ram speed”, and the “cooling water temperature” are associated with one another is one of heat treatment processes of a cast aluminum alloy, and refers to treatment in which Cu and Mg in an aluminum alloy are dissolved into the aluminum matrix. The billet heat treatment 113 and the hot extrusion treatment 114 in the manufacturing flow illustrated in FIG. 1 correspond to the solution treatment illustrated in FIG. 2.

<Relationship between Manufacturing Process and Metallographic Structure in Manufacturing Flow of Aluminum Alloy Extruded Profile>

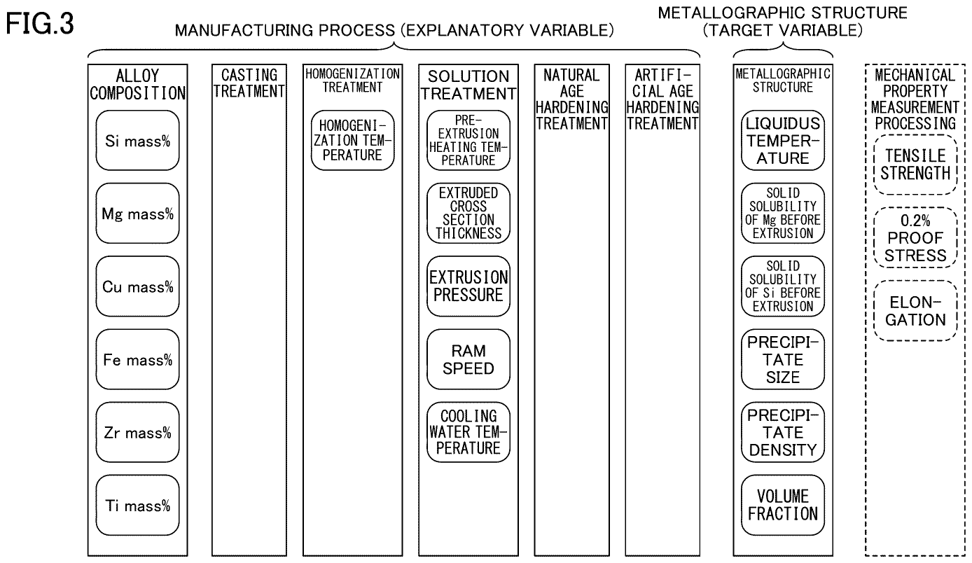

Next, the relationship between a manufacturing process and a metallographic structure in the manufacturing flow of the aluminum alloy extruded profile will be described. FIG. 3 is a diagram illustrating the relationship between the manufacturing process and the metallographic structure in the manufacturing flow of the aluminum alloy extruded profile. As described above, the mechanical property measurement processing 118 is performed to measure the mechanical properties of the aluminum alloy extruded profile manufactured according to the manufacturing flow.

The tensile strength, the 0.2% proof stress, and the elongation, which are the mechanical properties of the aluminum alloy extruded profile, are affected by the metallographic structure of the aluminum alloy extruded profile. Specifically, the mechanical properties of the aluminum alloy extruded profile are affected by the following data indicating the metallographic structure of the aluminum alloy extruded profile.
Liquidus temperature
Solid solubility of Mg before extrusion
Solid solubility of Si before extrusion
Precipitate size
Precipitate density
Volume fraction

Therefore, the manufacturing process search apparatus according to the first embodiment analyzes the relationship between the manufacturing process and the metallographic structure by using the manufacturing process as explanatory variables and the metallographic structure as target variables.

### <Outline of Processing Performed by Manufacturing Process Search Apparatus>

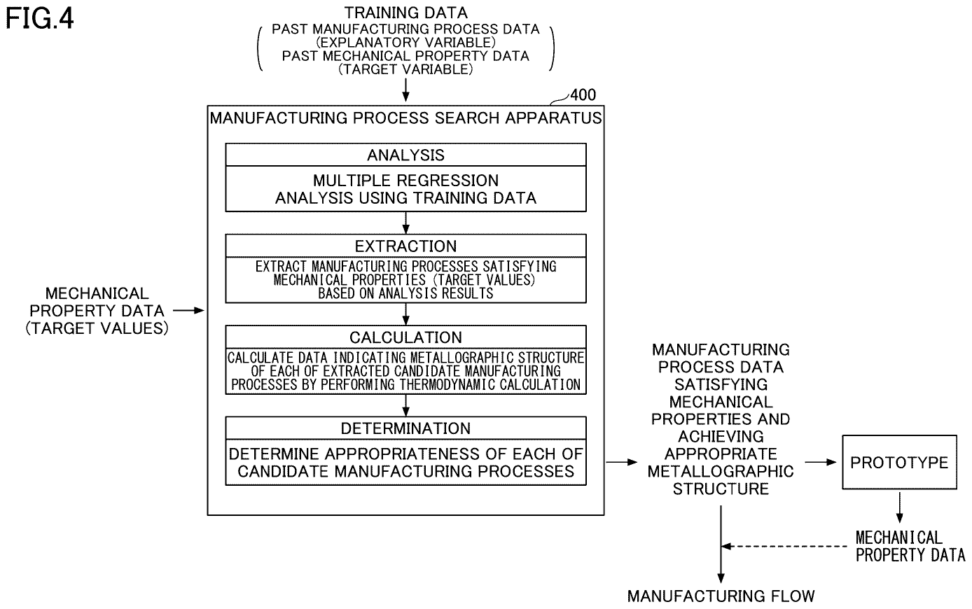

Next, an outline of processing performed by the manufacturing process search apparatus according to the first embodiment will be described. FIG. 4 is a diagram illustrating the outline of the processing performed by the manufacturing process search apparatus. As illustrated in FIG. 4, a manufacturing process search apparatus 400 searches for a manufacturing process through four phases.

A first phase is an analysis phase. In the analysis phase, the manufacturing process search apparatus 400 acquires past manufacturing process data and past mechanical property data as training data. Further, in the analysis phase, the manufacturing process search apparatus 400 performs multiple regression analysis by using the training data in which the acquired manufacturing process data are used as explanatory variables and the acquired mechanical property data are used as target variables.

A second phase is an extraction phase. In the extraction phase, the manufacturing process search apparatus 400 acquires mechanical property data target values. In the extraction phase, the manufacturing process search apparatus 400 extracts, as pieces of candidate manufacturing process data, pieces of manufacturing process data satisfying the acquired mechanical property data target values, based on analysis results of the multiple regression analysis.

A third phase is a calculation phase. In the calculation phase, the manufacturing process search apparatus 400 calculates data indicating a metallographic structure for each of the extracted pieces of candidate manufacturing process data by performing thermodynamic calculation.

Specifically, the solid solubility of an element and the phase fraction of precipitates in the aluminum alloy are identified by using a calculated equilibrium phase diagram based on the CALPHAD (CALculation of PHAse Diagrams, Computer Coupling of Phase Diagrams and Thermochemistry) method.

Note that the state of precipitates when an element is dissolved in the aluminum alloy during extrusion and then the precipitates are precipitated by artificial precipitation is predicted as follows.
Thermodynamic data and kinetic data determined based on the CALPHAD method are used.
Nucleation, growth, and coarsening of the precipitates are calculated by a numerical method based on the Langer-Schwartz theory and the Kampmann-Wagner method.

As a result, the size, the density, and the volume fraction of the precipitates can be predicted as the state of the precipitates.

When the state of the precipitates is predicted as described above, interface energy is input for both matrix growth and precipitate growth. Further, the sites where the precipitates are generated are assumed to be within the matrix, and the morphology of the precipitates is set to be needle-like or plate-like. In this manner, by performing simulation under more appropriate conditions, changes in the precipitates in the aluminum alloy over time can be accurately predicted.

A fourth phase is a determination phase. In the determination phase, the manufacturing process search apparatus 400 determines the appropriateness of each of the pieces of candidate manufacturing process data based on the calculated data indicating the metallographic structure. Thus, the manufacturing process search apparatus 400 can output manufacturing process data satisfying the mechanical property data target values and achieving an appropriate metallographic structure. That is, the manufacturing process search apparatus 400 can narrow down the pieces of candidate manufacturing process data.

Manufacturing process data narrowed down by the manufacturing process search apparatus 400 is used to manufacture a prototype, and the mechanical properties are verified. If the target mechanical characteristics are obtained, the corresponding manufacturing process data is applied to the manufacturing flow of the aluminum alloy extruded profile illustrated in FIG. 1.

### <Hardware Configuration of Manufacturing Process Search Apparatus>

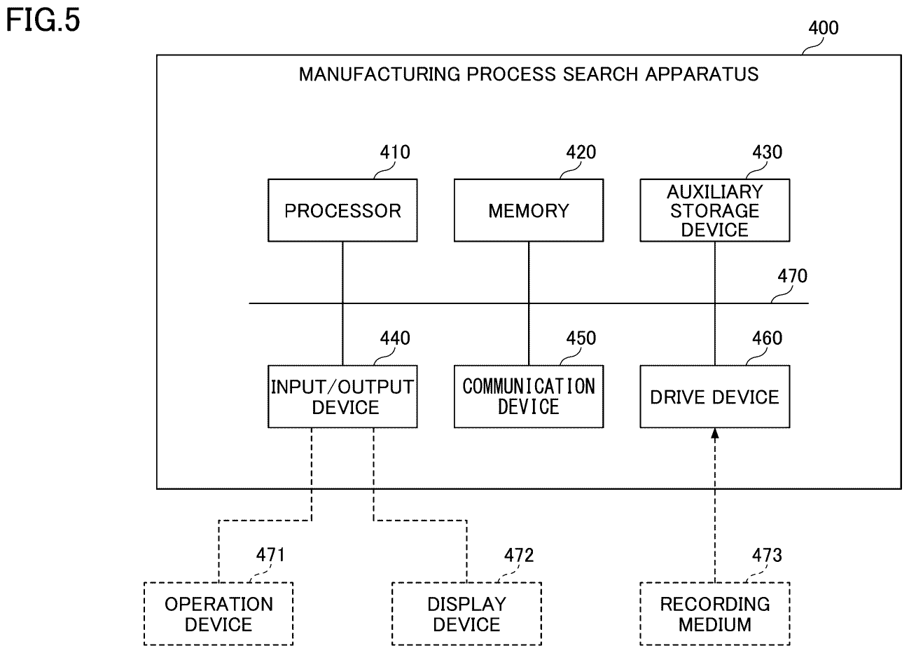

Next, a hardware configuration of the manufacturing process search apparatus 400 will be described. FIG. 5 is a diagram illustrating an example of the hardware configuration of the manufacturing process search apparatus. As illustrated in FIG. 5, the manufacturing process search apparatus 400 includes a processor 410, a memory 420, an auxiliary storage device 430, an input/output device 440, a communication device 450, and a drive device 460. The hardware components of the manufacturing process search apparatus 400 are connected to one another via a bus 470.

The processor 410 includes various computing devices such as a central processing unit (CPU) and a graphics processing unit (GPU). The processor 410 loads various programs into the memory 420 and executes the various programs. Note that the various programs include, for example, a program such as a material manufacturing process search program (hereinafter simply referred to as a “manufacturing process search program”), which will be described later.

The memory 420 includes a main storage device such as a read-only memory (ROM) and a random-access memory (RAM). The processor 410 and the memory 420 form what is known as a computer. The computer implements the above-described various functions by causing the processor 410 to execute the various programs loaded into the memory 420.

The auxiliary storage device 430 stores various programs, and various data used when the various programs are executed by the processor 410. For example, a training data storage unit 650, which will be described later, is implemented by the auxiliary storage device 430.

The input/output device 440 is a connection device that connects to an operation device 471 and a display device 472, which are examples of user interface devices. The communication device 450 is a communication device for communicating with an external apparatus (not illustrated) via a network.

The drive device 460 is a device into which a recording medium 473 is set. The recording medium 473 includes a medium for optically, electrically, or magnetically recording information, such as a CD-ROM, a flexible disk, or a magneto-optical disc. The recording medium 473 may include a semiconductor memory or the like for electrically recording information, such as a ROM or a flash memory.

The various programs installed in the auxiliary storage device 430 are installed by setting the recording medium 473 that is distributed into the drive device 460, and reading the various programs recorded in the recording medium 473 by the drive device 460, for example. Alternatively, the various programs installed in the auxiliary storage device 430 may be installed by being downloaded from the network via the communication device 450.

### <Functional Configuration of Manufacturing Process Search Apparatus>

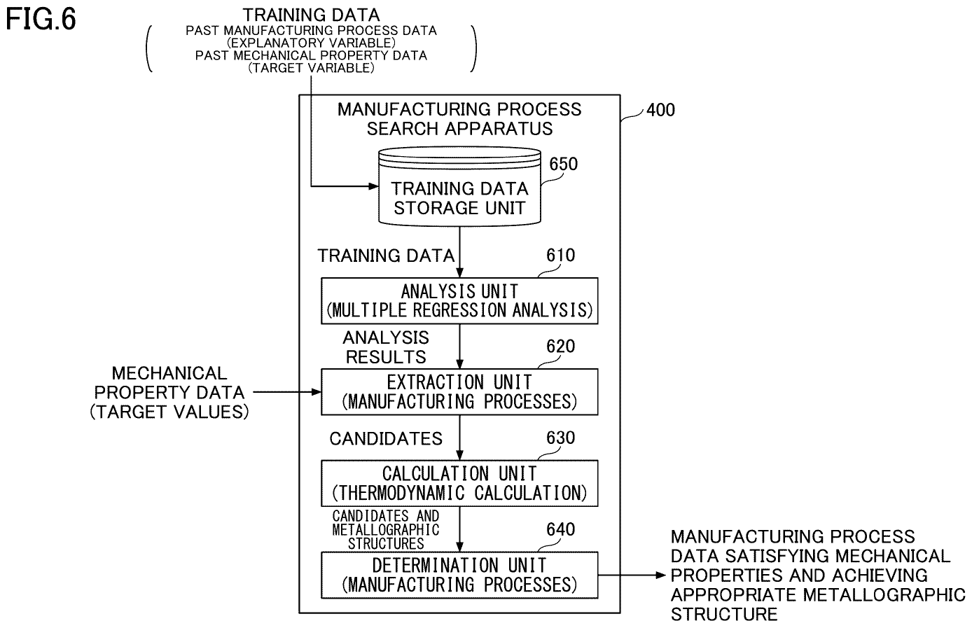

Next, a functional configuration of the manufacturing process search apparatus 400 will be described. FIG. 6 is a diagram illustrating an example of the functional configuration of the manufacturing process search apparatus. As described above, the manufacturing process search program is installed in the manufacturing process search apparatus 400. The manufacturing process search apparatus 400 functions as an analysis unit 610, an extraction unit 620, a calculation unit 630, and a determination unit 640 by executing the manufacturing process search program.

The analysis unit 610 functions in the analysis phase. The analysis unit 610 reads the training data stored in the training data storage unit 650. The training data storage unit 650 stores:
past manufacturing process data applied to the manufacturing flow illustrated in FIG. 1; and
past mechanical property data of an aluminum alloy extruded profile manufactured according to the manufacturing flow to which the corresponding past manufacturing process data is applied.

The analysis unit 610 performs multiple regression analysis by using the manufacturing process data included in the read training data as explanatory variables and the mechanical property data as target variables. The analysis unit 610 sends analysis results of the multiple regression analysis to the extraction unit 620.

The extraction unit 620 functions in the extraction phase. The extraction unit 620 acquires mechanical property data target values. The extraction unit 620 extracts manufacturing process data satisfying the acquired mechanical property data target values, based on the analysis results of the multiple regression analysis.

Specifically, the extraction unit 620 exhaustively generates combinations of values of elements of manufacturing process data by varying the values of the elements of the manufacturing process data with a predetermined step size. The extraction unit 620 inputs the exhaustively generated combinations into the analysis results of the multiple regression analysis, and predicts mechanical property data for each of the combinations. The extraction unit 620 determines whether the predicted mechanical property data satisfies the mechanical property data target values. If the extraction unit 620 determines that mechanical property data predicted for a corresponding combination satisfies the mechanical property data target values, the extraction unit 620 extracts manufacturing process data of the corresponding combination as candidate manufacturing process data. The extraction unit 620 sends the extracted candidate manufacturing process data to the calculation unit 630.

The calculation unit 630 functions in a calculation phase. The calculation unit 630 calculates data indicating metallographic structures for respective pieces of candidate manufacturing process data sent from the extraction unit 620 by performing thermodynamic calculation for the pieces of candidate manufacturing process data. The calculation unit 630 sends the calculated data indicating the metallographic structures and the pieces of candidate manufacturing process data to the determination unit 640.

The determination unit 640 functions in the determination phase. For each of the pieces of candidate manufacturing process data sent from the calculation unit 630, the determination unit 640 determines whether data indicating a metallographic structure satisfies conditions for achieving the mechanical property data target values. If the determination unit 640 determines that data indicating a metallographic structure satisfies the conditions for achieving the mechanical property data target values, the determination unit 640 outputs corresponding candidate manufacturing process data. Conversely, if the determination unit 640 determines that data indicating a metallographic structure does not satisfy the conditions for achieving the mechanical property data target values, the determination unit 640 does not output corresponding candidate manufacturing process data. Therefore, the pieces of candidate manufacturing process data output from the determination unit 640 are narrowed down to manufacturing process data satisfying the mechanical property data target values and achieving an appropriate metallographic structure.

### <Specific Example of Processing Performed by Each Unit of Manufacturing Process Search Apparatus>

Next, a specific example of processing performed by each of the analysis unit 610, extraction unit 620, the calculation unit 630, and determination unit 640 of the manufacturing process search apparatus 400 will be described.

(1) Specific Example of Processing Performed by the Analysis unit

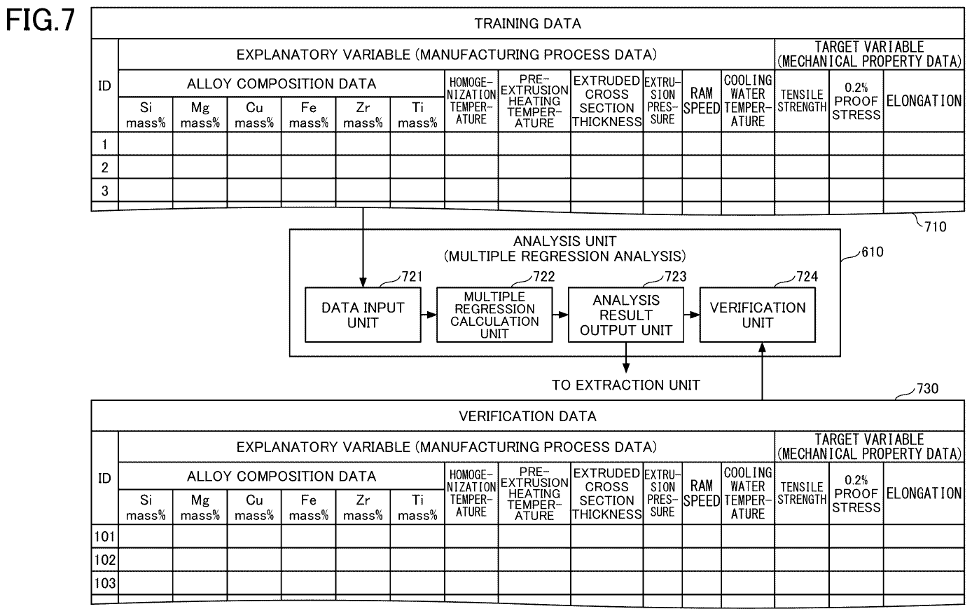

First, a specific example of processing performed by the analysis unit 610 of the manufacturing process search apparatus 400 will be described. FIG. 7 is a first diagram illustrating the specific example of the processing performed by the analysis unit.

As illustrated in FIG. 7, training data 710 read from the training data storage unit 650 by the analysis unit 610 includes, as information items, “ID”, “explanatory variable”, and “target variable”.

The “ID” stores an identification number for identifying a combination of “explanatory variable” and “target variable” included in the training data 710.

The “explanatory variable” includes “alloy composition data”, “homogenization temperature”, “pre-extrusion heating temperature”, “extruded cross section thickness”, “extrusion pressure”, “ram speed”, and “cooling water temperature”. The “alloy composition data” further includes “Si mass %”, “Mg mass %”, “Cu mass %”, “Fe mass %”, “Zr mass %”, and “Ti mass %”, which are alloy composition data included in past manufacturing process data applied to the manufacturing flow illustrated in FIG. 1. Note that data stored in each item included in the information item “explanatory variable” has already been described and thus a description thereof will be omitted.

The “target variable” includes “tensile strength”, “0.2% proof stress”, and “elongation”, which are mechanical properties of an aluminum alloy extruded profile manufactured according to the manufacturing flow illustrated in FIG. 1 to which the past manufacturing process data is applied. Note that data stored in each item included in the information item “target variable” has already been described and thus a description thereof will be omitted.

As illustrated in FIG. 7, the analysis unit 610 further includes a data input unit 721, a multiple regression calculation unit 722, an analysis result output unit 723, and a verification unit 724.

The data input unit 721 inputs part of training data stored in the training data storage unit 650 into the multiple regression calculation unit 722 as training data 710.

The multiple regression calculation unit 722 performs multiple regression analysis by using manufacturing process data as explanatory variables and mechanical property data as target variables, which is included in the training data 710 input by the data input unit 721.

The analysis result output unit 723 sends analysis results of the multiple regression analysis obtained by the multiple regression calculation unit 722 to the extraction unit 620 and the verification unit 724.

The verification unit 724 reads out, as verification data 730, training data, stored in the training data storage unit 650, that is training data other than the training data read as the training data 710. Information items included in the verification data 730 are the same as the information items included in the training data 710, and thus a description thereof will be omitted.

The verification unit 724 inputs, as explanatory variables, manufacturing process data included in the verification data 730 into the analysis results of the multiple regression analysis so as to predict mechanical property data for the corresponding manufacturing process data. The verification unit 724 verifies the validity of the analysis results of the multiple regression analysis by determining whether error between the predicted mechanical property data and mechanical property data included in the verification data 730 is less than or equal to a predetermined threshold.

Specifically, if the verification unit 724 determines that the error between the predicted mechanical property data and the mechanical property data included in the verification data 730 is less than or equal to the predetermined threshold, the verification unit 724 determines that the analysis results of the multiple regression analysis are valid.

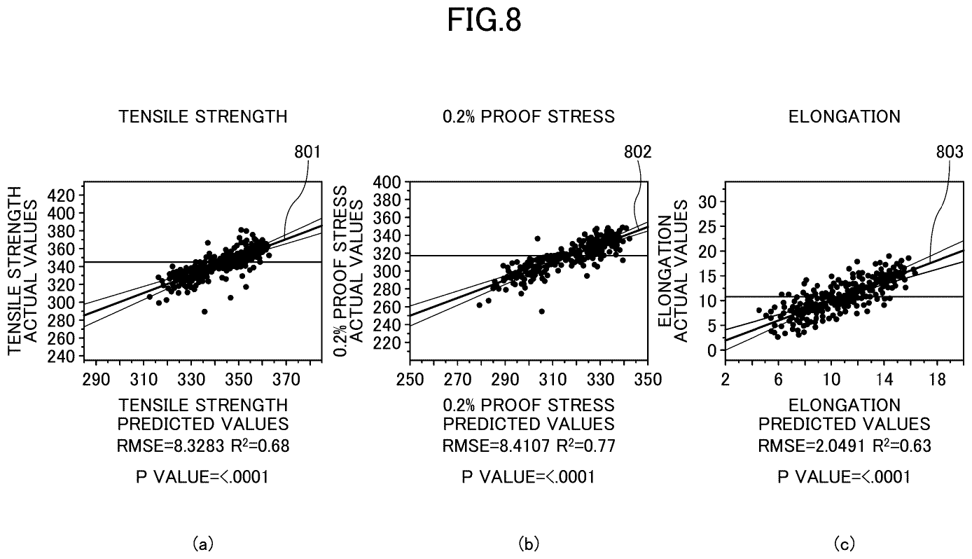

FIG. 8 is a diagram illustrating a verification example of the analysis results of the multiple regression analysis. FIG. 8(a) is a graph illustrating the relationship between tensile strength of the predicted mechanical property data and tensile strength data included in the verification data 730. In FIG. 8(a), the horizontal axis represents the predicted tensile strength, and the vertical axis represents the tensile strength included in the verification data 730. As errors between the predicted tensile strength and the tensile strength included in the verification data 730 decrease, the variation of plotted points with respect to a straight line 801 becomes smaller. Conversely, as errors between the predicted tensile strength and the tensile strength included in the verification data 730 increase, the variation of the plotted points with respect to the straight line 801 becomes larger.

As illustrated in FIG. 8(a), with respect to the tensile strength, the root mean square error (RMSE) was 8.3283, the coefficient of determination (R²) was 0.68, and the P value was less than 0.0001. In other words, it was verified that the analysis results of the multiple regression analysis was generally satisfactory for the tensile strength.

FIG. 8(b) is a graph illustrating the relationship between 0.2% proof stress of the predicted mechanical property data and 0.2% proof stress included in the verification data 730. In FIG. 8(b), the horizontal axis represents the predicted 0.2% proof stress, and the vertical axis represents the 0.2% proof stress included in the verification data 730. As errors between the predicted 0.2% proof stress and the 0.2% proof stress included in the verification data 730 decrease, the variation of plotted points with respect to a straight line 802 becomes smaller. Conversely, as errors between the predicted 0.2% proof stress and the 0.2% proof stress included in the verification data 730 increase, the variation of the plotted points with respect to the straight line 802 becomes larger.

As illustrated in FIG. 8(b), with respect to the 0.2% proof stress, the root mean square error (RMSE) was 8.4107, the coefficient of determination (R²) was 0.77, and the P value was less than 0.0001. In other words, it was verified that the analysis results of the multiple regression analysis were generally satisfactory for the 0.2% proof stress.

FIG. 8(c) is a graph illustrating the relationship between elongation of the predicted mechanical property data and elongation included in the verification data 730. In FIG. 8(c), the horizontal axis represents the predicted elongation, and the vertical axis represents the elongation included in the verification data 730. As errors between the predicted elongation and the elongation included in the verification data 730 decrease, the variation of plotted points with respect to a straight line 803 becomes smaller. Conversely, as errors between the predicted elongation and the elongation included in the verification data 730 increase, the variation of the plotted points with respect to the straight line 802 becomes larger.

As illustrated in FIG. 8(c), the root mean square error (RMSE) of the elongation was 2.0491, the coefficient of determination (R²) was 0.63, and the P value was less than 0.0001. In other words, it was verified that the analysis results of the multiple regression analysis were generally satisfactory for the elongation.

### (2) Specific Example of Processing Performed by the Extraction Unit

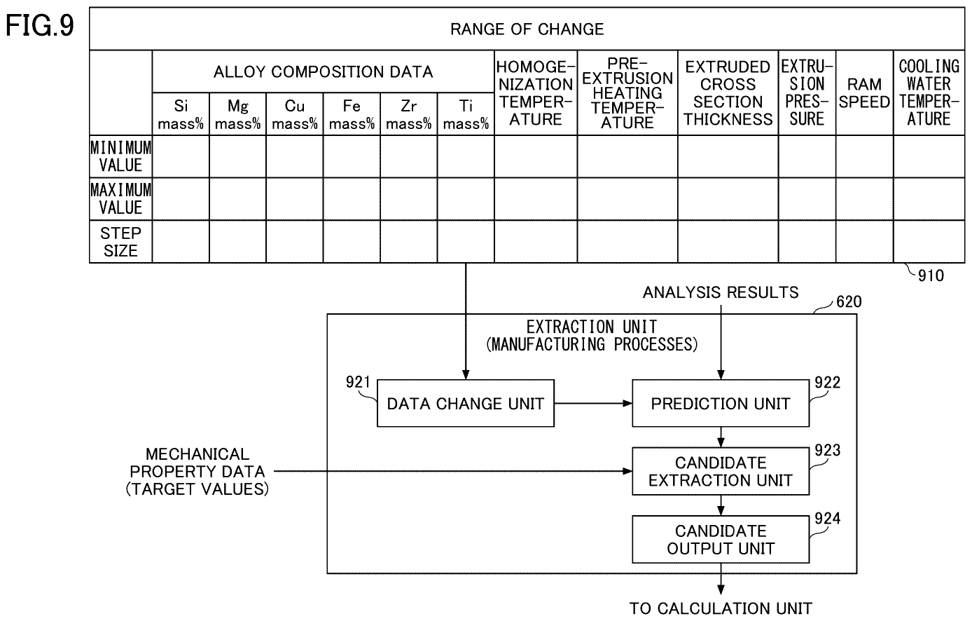

Next, a specific example of processing performed by the extraction unit 620 of the manufacturing process search apparatus 400 will be described. FIG. 9 is a diagram illustrating the specific example of the processing performed by the extraction unit.

As illustrated in FIG. 9, the extraction unit 620 includes a data change unit 921, a prediction unit 922, a candidate extraction unit 923, and a candidate output unit 924.

The data change unit 921 exhaustively generates combinations of values of elements of manufacturing process data by changing the values of the elements of the manufacturing process data with a predetermined step size. As illustrated in change range data 910 of FIG. 9, a minimum value, a maximum value, and a step size are preset for each of the elements of the manufacturing process data. The data change unit 921 exhaustively generates various combinations of values of the elements of the manufacturing process data by changing each of the values of the elements within a range between the minimum value and the maximum value illustrated in the change range data 910 by using the step size illustrated in the change range data 910.

The data change unit 921 sends the exhaustively generated combinations of the values of the elements of the manufacturing process data to the prediction unit 922.

The analysis results of the multiple regression analysis sent from the analysis unit 610 are set in the prediction unit 922. Upon the combinations of the values of the elements of the manufacturing process data being sent from the data change unit 921, the prediction unit 922 inputs the combinations of the values of the elements of the manufacturing process data into the analysis results of the multiple regression analysis. Then, the prediction unit 922 predicts mechanical property data for each of the combinations. The prediction unit 922 associates the predicted mechanical property data with each of the combinations of the values of the elements of the manufacturing process data, and sends the predicted mechanical property data to the candidate extraction unit 923.

The candidate extraction unit 923 acquires mechanical property data target values. The candidate extraction unit 923 compares the mechanical property data sent from the prediction unit 922 with the acquired mechanical property data targe values. If the candidate extraction unit 923 determines that:
tensile strength included in mechanical property data sent from the prediction unit 922 is greater than tensile strength included in the acquired mechanical property data target values;
0.2% proof stress included in the mechanical property data sent from the prediction unit 922 is greater than 0.2% proof stress included in the acquired mechanical property data target values; and
elongation included in the mechanical property data sent from the prediction unit 922 is greater than elongation included in the acquired mechanical property data target values,
the candidate extraction unit 923 extracts a corresponding combination of values of elements of manufacturing process data, which corresponds to the mechanical property data, as candidate manufacturing process data.

The candidate extraction unit 923 sends the extracted candidate manufacturing process data to the candidate output unit 924.

The candidate output unit 924 sends the extracted candidate manufacturing process data, sent from the candidate extraction unit 923, to the calculation unit 630.

### (3) Specific Example of Processing Performed by the Calculation Unit

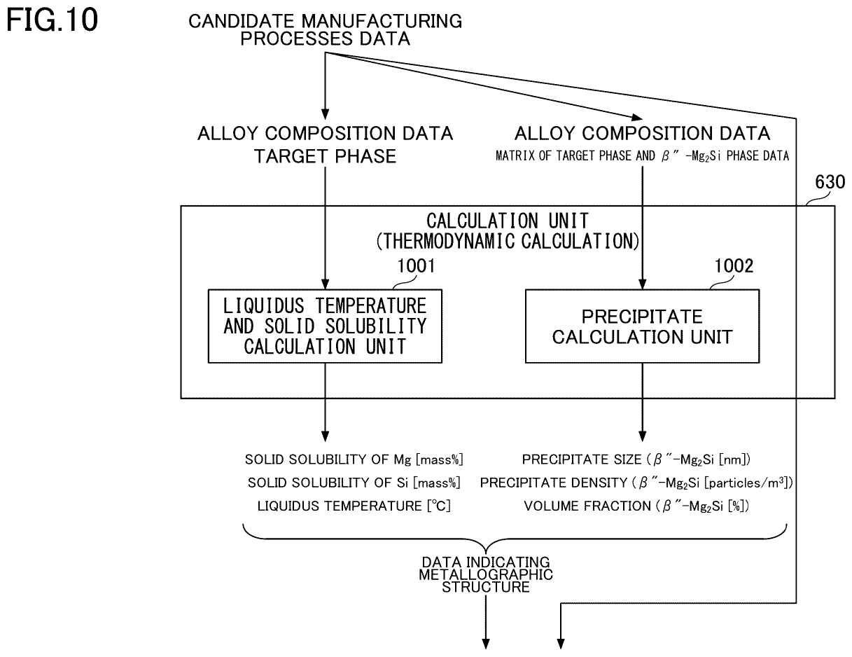

Next, a specific example of processing performed by the calculation unit 630 of the manufacturing process search apparatus 400 will be described. FIG. 10 is a first diagram illustrating the specific example of the processing performed by the calculation unit.

The calculation unit 630 is general-purpose thermodynamic calculation software. For example, thermodynamic calculation software such as Thermo-Calc is used.

The thermodynamic calculation software identifies the solid solubility of an element and the phase fraction of precipitates in an aluminum alloy by using a calculated equilibrium phase diagram based on the CALPHAD method.

In addition, the thermodynamic calculation software predicts the state of precipitates when an element is dissolved in an aluminum alloy during extrusion and the precipitates are precipitated by artificial precipitation as follows.
Thermodynamic data and kinetic data determined based on the CALPAD method are used.
Nucleation, growth, and coarsening of the precipitates are calculated by a numerical method based on the Langer-Schwartz theory and the Kampmann-Wagner method.

As a result, the size, the density, and the volume fraction of the precipitates can be predicted as the state of the precipitates.

When the state of the precipitates is predicted as described above, interface energy is input for both matrix growth and precipitate growth. Further, the sites where the precipitates are generated are assumed to be within the matrix, and the morphology of the precipitates is set to be needle-like or plate-like. In this manner, by performing simulation under more appropriate conditions, changes in the precipitates in the aluminum alloy over time can be accurately predicted.

FIG. 10 schematically illustrates the functions of the above-described thermodynamic calculation software. A liquidus temperature and solid solubility calculation unit 1001 and a precipitate calculation unit 1002 are used.

The example of FIG. 10 illustrates that the liquidus temperature and solid solubility calculation unit 1001 is used to calculate a liquidus temperature and a solid solubility for the candidate manufacturing process data sent from the extraction unit 620.

Specifically, alloy composition data included in the candidate manufacturing process data and information about a target phase are input into the liquidus temperature and solid solubility calculation unit 1001, and as a result, the following are output.
Solid solubility of Mg [mass %]
Solid solubility of Si [mass &]
Liquidus temperature [°C]Further, the precipitate calculation unit 1002 is used to calculate a precipitate size, a precipitate density, and a volume fraction for the candidate manufacturing process data sent from the extraction unit 620.

Specifically, the alloy composition data included in the candidate manufacturing process data, a matrix of the target phase, and β″-Mg₂Si phase data are input into the precipitate calculation unit 1002, and as a result, the following are output.
Precipitate (β″-Mg₂Si) size (average particle size) [nm]
Precipitate (β″-Mg₂Si) density (number density) [particles/m³]
Precipitate (β″-Mg₂Si) volume fraction [volume %]

Thus, the calculation unit 630 can associate the candidate manufacturing process data sent from the extraction unit 620 with data indicating a metallographic structure, which includes the liquidus temperature, the solid solubilities, the precipitate size, the precipitate density, and the volume fraction, and send the candidate manufacturing process data associated with the data indicating the metallographic structure to the determination unit 640.

### (4) Specific Example of Processing Performed by the Determination Unit

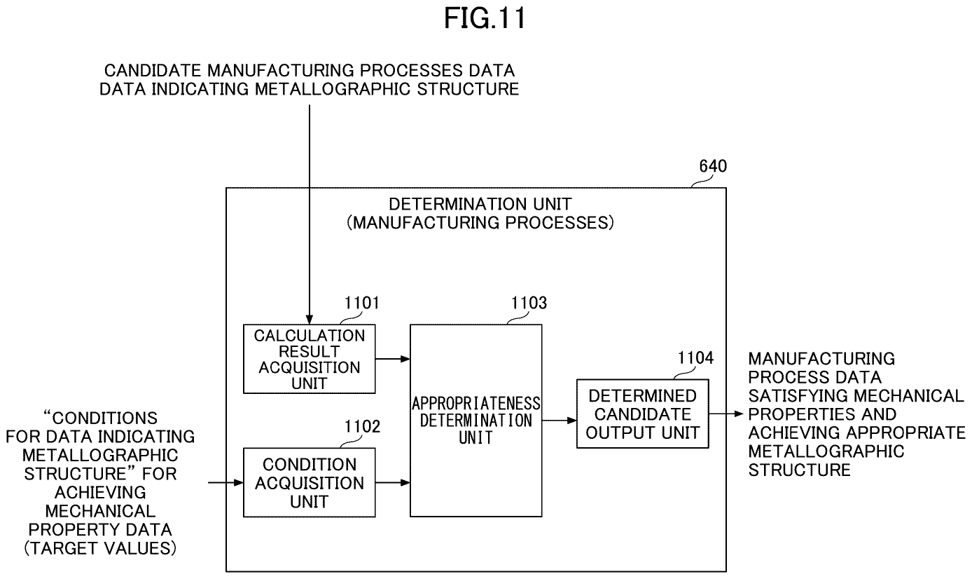

Next, a specific example of processing performed by the determination unit 640 of the manufacturing process search apparatus 400 will be described. FIG. 11 is a diagram illustrating the specific example of the processing performed by the determination unit.

As illustrated in FIG. 11, the determination unit 640 includes a calculation result acquisition unit 1101, a condition acquisition unit 1102, an appropriateness determination unit 1103, and a determined candidate output unit 1104.

The calculation result acquisition unit 1101 acquires, from the calculation unit 630, the candidate manufacturing process data and the data indicating the corresponding metallographic structure. Further, the calculation result acquisition unit 1101 sends the acquired candidate manufacturing process data and the data indicating the corresponding metallographic structure to the appropriateness determination unit 1103.

The condition acquisition unit 1102 acquires “conditions for data indicating metallographic structure” for achieving the mechanical property data target values. The condition acquisition unit 1102 sends the acquired “conditions for data indicating metallographic structure” to the appropriateness determination unit 1103.

The appropriateness determination unit 1103 determines whether the data indicating the metallographic structure sent from the calculation result acquisition unit 1101 satisfies the “conditions for data indicating metallographic structure” sent from the condition acquisition unit 1102. If the appropriateness determination unit 1103 determines that the data indicating the metallographic structure sent from the calculation result acquisition unit 1101 satisfies the “conditions for data indicating metallographic structure” sent from the condition acquisition unit 1102, the appropriateness determination unit 1103 determines that the corresponding candidate manufacturing process data is appropriate. Conversely, if the appropriateness determination unit 1103 determines that the data indicating the metallographic structure sent from the calculation result acquisition unit 1101 does not satisfy the “conditions for data indicating metallographic structure” sent from the condition acquisition unit 1102, the appropriateness determination unit 1103 determines that the corresponding candidate manufacturing process data is not appropriate.

The appropriateness determination unit 1103 sends the candidate manufacturing process data determined to be appropriate to the determined candidate output unit 1104.

The determined candidate output unit 1104 outputs the candidate manufacturing process data, sent from the appropriateness determination unit 1103, as manufacturing process data satisfying the mechanical property data target values and achieving an appropriate metallographic structure.

### <Flow of Manufacturing Process Search Processing>

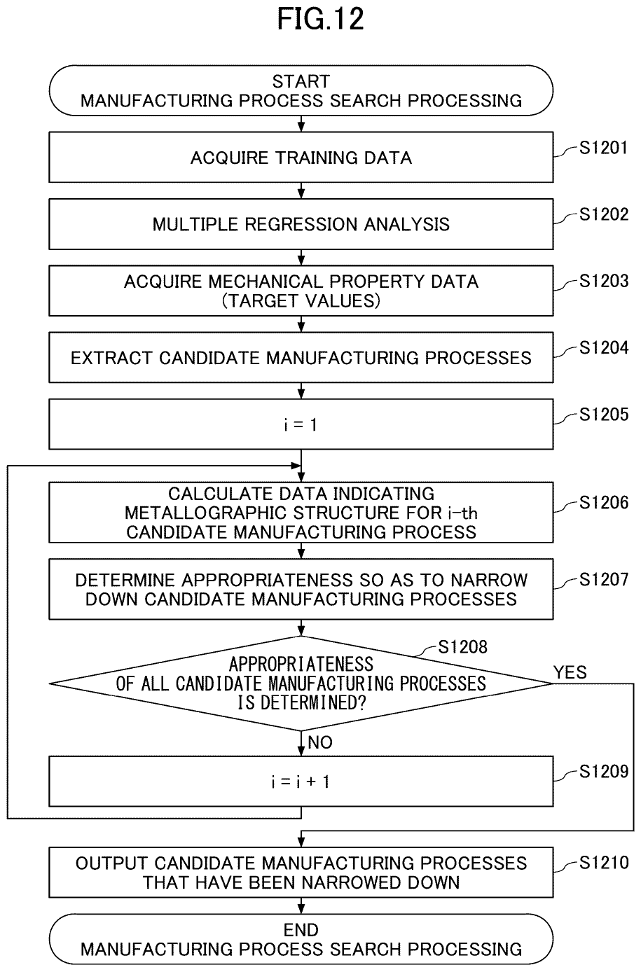

Next, a flow of manufacturing process search processing performed by the manufacturing process search apparatus 400 will be described. FIG. 12 is a flowchart illustrating the flow of the manufacturing process search processing.

In step S1201, the manufacturing process search apparatus 400 acquires training data.

In step S1202, the manufacturing process search apparatus 400 uses the training data to perform multiple regression analysis, and outputs analysis results of the multiple regression analysis.

In step S1203, the manufacturing process search apparatus 400 acquires mechanical property data target values.

In step S1204, the manufacturing process search apparatus 400 extracts pieces of candidate manufacturing process data with respect to which mechanical property data predicted based on the analysis results of the multiple regression analysis satisfies the acquired mechanical property data target values.

In step S1205, the manufacturing process search apparatus 400 inputs “1” to a counter i that counts the extracted pieces of candidate manufacturing process data.

In step S1206, the manufacturing process search apparatus 400 calculates data indicating a metallographic structure by performing thermodynamic calculation for the i-th candidate manufacturing process data among the extracted pieces of manufacturing process data.

In step S1207, the manufacturing process search apparatus 400 determines whether the data indicating the metallographic structure calculated for the i-th candidate manufacturing process data satisfies the “conditions for data indicating metallographic structure” for achieving the mechanical property data target values. Thus, the manufacturing process search apparatus 400 determines the appropriateness of the i-th candidate manufacturing process data so as to narrow down the pieces of candidate manufacturing process data.

In step S1208, the manufacturing process search apparatus 400 determines whether the appropriateness of all the pieces of candidate manufacturing process data is determined. In step S1208, if the manufacturing process search apparatus 400 determines that there is candidate manufacturing process data whose appropriateness has not been determined (NO in step S1208), the manufacturing process search apparatus 400 causes the processing to proceed to step S1209.

In step S1209, the manufacturing process search apparatus 400 increments the counter i, and causes the processing to proceed to step S1206.

Conversely, in step S1208, if the manufacturing process search apparatus 400 determines that the appropriateness of all the pieces of candidate manufacturing process data is determined, the manufacturing process search apparatus 400 causes the processing to proceed to step S1210.

In step S1210, the manufacturing process search apparatus 400 outputs candidate manufacturing process data that has been determined to be appropriate, and then ends the manufacturing process search processing.

### <Example>

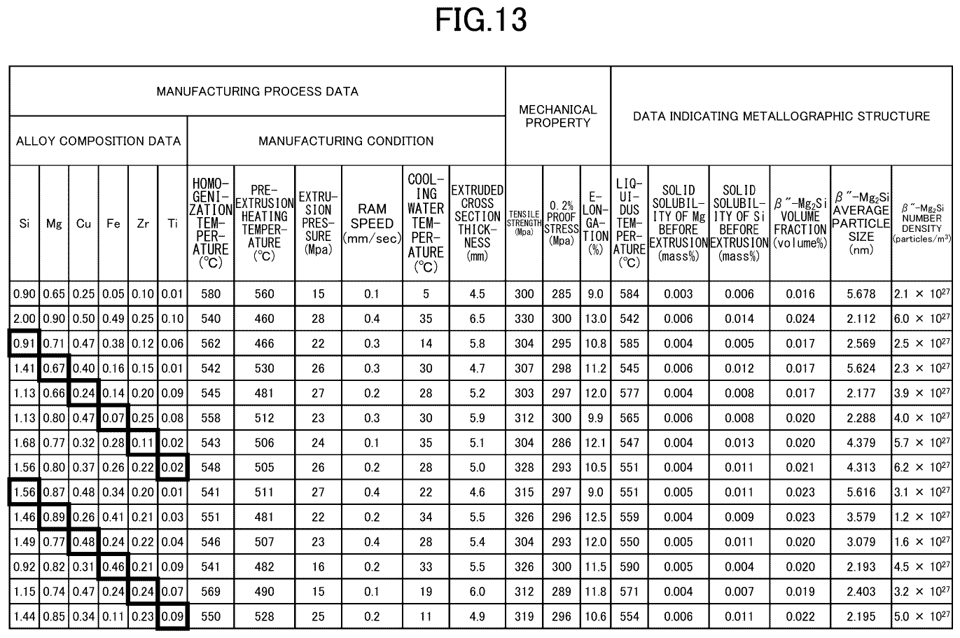

Next, manufacturing process search processing performed by the manufacturing process search apparatus 400 according to an example will be described. FIG. 13 is a diagram illustrating an example of search results obtained by the manufacturing process search processing, and is a diagram illustrating search results when mechanical property data target values and “conditions for data indicating metallographic structure” are set as follows.

The mechanical property data target values are set as follows.
Tensile strength: 300 [MPa] or more
0.2% Proof stress: 270 [MPa] or more
Elongation: 9.0 [%] or more
   “Conditions for data indicating metallographic structure” are set as follows.
Liquidus temperature: 560 [°C] or more
Solid solubility of Mg before extrusion: 0.005 [mass %] or more
Solid solubility of Si before extrusion: 0.01 [mass %] or more
Precipitate (β″-Mg₂Si) size (average particle size): 50 [nm] or more and 300 nm or less
Precipitate (β″-Mg₂Si) density (number density): 6.0×1027 [particles/m³] or more
Precipitate (β″-Mg₂Si) volume fraction: 10 [volume %] or more

In the example of FIG. 13, each of fourteen pieces of manufacturing process data is output as manufacturing process data satisfying the mechanical property data target values and achieving an appropriate metallographic structure.

### <Summary>

As is clear from the above description, the manufacturing process search apparatus 400 according to the first embodiment includes:
the analysis unit 610 configured to analyze a relationship between manufacturing process data of an aluminum alloy extruded profile and mechanical property data of the aluminum alloy extruded profile by using multiple regression analysis;
the extraction unit 620 configured to extract pieces of candidate manufacturing process data for the aluminum alloy extruded profile satisfying mechanical property data target values, based on analysis results of the multiple regression analysis obtained by the analysis unit 610;
the calculation unit 630 configured to calculate data indicating a metallographic structure, which is data affecting mechanical property data, by performing thermodynamic calculation for each of the extracted pieces of candidate manufacturing process data; and
the determination unit 640 configured to determine whether the data indicating the metallographic structure, calculated by the calculation unit 630 performing the thermodynamic calculation for each of the extracted pieces of candidate manufacturing process date, satisfies “conditions for data indicating metallographic structure” for achieving the mechanical property data target values. The determination unit 640 determines appropriateness of each of the pieces of candidate manufacturing process data based on whether the data indicating the metallographic structure satisfies the “conditions for data indicating metallographic structure”.

As described above, the manufacturing process search apparatus 400 according to the first embodiment can output manufacturing process data satisfying the mechanical property data target values and achieving an appropriate metallographic structure. That is, even if there are many manufacturing processes that can achieve an aluminum alloy extruded profile satisfying desired mechanical characteristics, the manufacturing processes can be narrowed down. As a result, according to the first embodiment, it is possible to efficiently search for a manufacturing process that can achieve an aluminum alloy extruded profile satisfying desired mechanical characteristics.

### Second Embodiment

In the first embodiment, tensile strength, 0.2% proof stress, and elongation are used as mechanical property data, but the mechanical property data is not limited thereto, and other mechanical property data may be used.

Further, in the example of the first embodiment, the mechanical property data target values are set to tensile strength ≥300 MPa, 0.2% proof stress ≥270 MPa, and elongation ≥9.0%, but the mechanical property data target values are not limited thereto.

Further, in the above-described first embodiment, the homogenization temperature, the pre-extrusion heating temperature, the extruded cross section thickness, the ram speed, and the cooling water temperature are used as manufacturing conditions, but the manufacturing conditions are not limited thereto, and other manufacturing conditions may be used.

Further, in the first embodiment, the data change unit 921 exhaustively generates combinations of values of elements of manufacturing process data, but the method of generating combinations of values of elements of manufacturing process data by the data change unit 921 is not limited thereto.

Further, in the first embodiment, the solid solubility, the liquidus temperature, the precipitate size, the precipitate density, and the volume fraction are calculated as data indicating a metallographic structure, but the data indicating the metallographic structure is not limited thereto, and any other data indicating a metallographic structure may be calculated.

Further, in the first embodiment, “conditions for data indicating metallographic structure” are set as follows, but the “conditions for data indicating metallographic structure” are not limited thereto.
Liquidus temperature: 560 [°C] or more
Solid solubility of Mg before extrusion: 0.005 [mass %] or more
Solid solubility of Si before extrusion: 0.01 [mass %] or more
Size (average particle size) of precipitates (β″-Mg₂Si): 50 [nm] or more and 300 nm or less
Density (number density) of precipitates (β″-Mg₂Si): 6.0×1027 [particles/m³] or more
Volume fraction of precipitates (β″-Mg₂Si): 10 [volume %] or more

### Third Embodiment

In the above-described first embodiment, an example in which the design target material is an alloy material and is specifically a wrought aluminum alloy material has been described. However, the design target material is not limited to the wrought aluminum alloy material and may be a cast aluminum alloy material. Hereinafter, the third embodiment will be described mainly with respect to differences from the first embodiment.

<Specific Example of Processing Performed by Analysis unit of Manufacturing Process Search Apparatus>

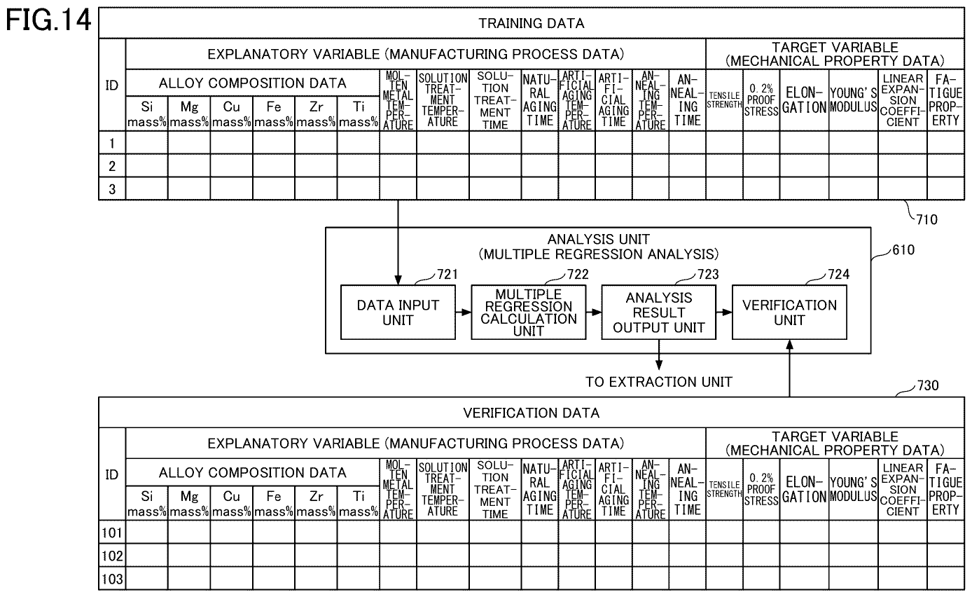

First, a specific example of processing performed by the analysis unit 610 of the manufacturing process search apparatus 400 will be described. FIG. 14 is a second diagram illustrating the specific example of the processing performed by the analysis unit.

Similarly to FIG. 7, as illustrated in FIG. 14, training data 710 read from the training data storage unit 650 by the analysis unit 610 includes, as information items, “ID”, “explanatory variable”, and “target variable”.

The “ID” stores an identification number for identifying a combination of “explanatory variable” and “target variable” included in the training data 710.

The “explanatory variable” includes, in the case of a cast aluminum alloy material, “alloy composition data”, “molten metal temperature” during pouring, “solution treatment temperature”, “solution treatment time”, “natural aging time”, “artificial aging temperature”, “artificial aging time”, “annealing temperature”, and “annealing time”. The “alloy composition data” includes “Si mass %”, “Mg mass %”, “Cu mass %”, “Fe mass %”, “Zr mass %”, and “Ti mass %”.

The “target variable” includes “tensile strength”, “0.2% proof stress”, “elongation”, “Young's modulus”, “linear expansion coefficient”, and “fatigue property”, which are mechanical properties of the cast aluminum alloy material.

Note that the units included in the analysis unit 610 illustrated in FIG. 14 have already been described in the above first embodiment, and thus a description thereof will be omitted. Further, verification data 730 is the same as the training data 710, and thus a description thereof will be omitted.

### <Specific Example of Processing Performed by Calculation Unit of Manufacturing Process Search Apparatus>

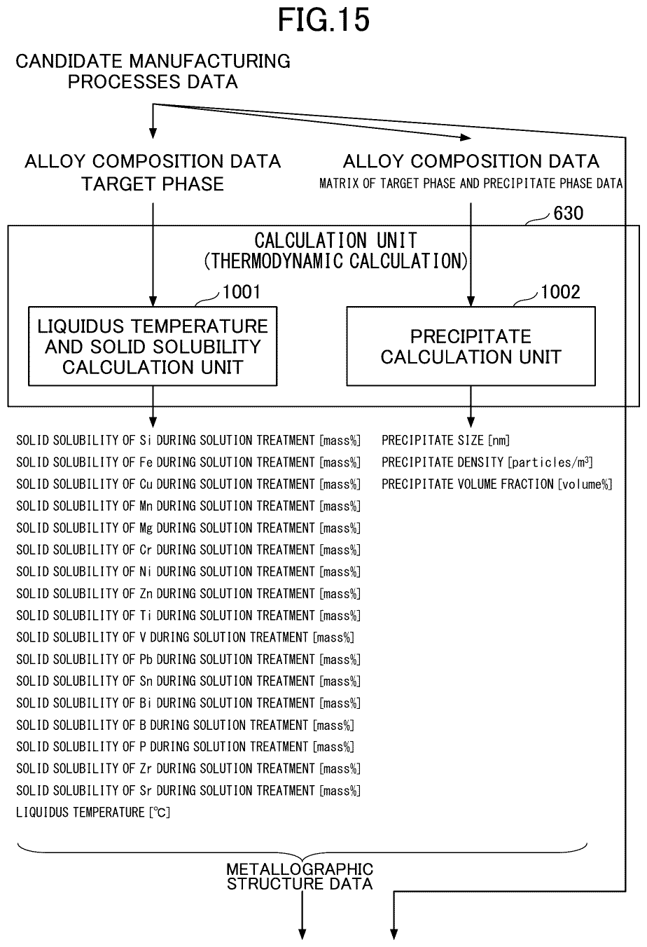

Next, a specific example of processing performed by the calculation unit 630 of the manufacturing process search apparatus 400 will be described. FIG. 15 is a second diagram illustrating the specific example of the processing performed by the calculation unit.

As illustrated in FIG. 15, in the case of the cast aluminum alloy material, the liquidus temperature and solid solubility calculation unit 1001 outputs the following.
Solid solubility of Si during solution treatment [mass %]
Solid solubility of Fe during solution treatment [mass %]
Solid solubility of Cu during solution treatment [mass %]
Solid solubility of Mn during solution treatment [mass %]
Solid solubility of Mg during solution treatment [mass %]
Solid solubility of Or during solution treatment [mass %]
Solid solubility of Ni during solution treatment [mass %]
Solid solubility of Zn during solution treatment [mass %]
Solid solubility of Ti during solution treatment [mass %]
Solid solubility of V during solution treatment [mass %]
Solid solubility of Pb during solution treatment [mass %]
Solid solubility of Sn during solution treatment [mass %]
Solid solubility of Bi during solution treatment [mass %]
Solid solubility of B during solution treatment [mass %]
Solid solubility of P during solution treatment [mass %]
Solid solubility of Zr during solution treatment [mass %]
Solid solubility of Sr during solution treatment [mass %]
Liquidus temperature [°C]

Further, in the case of the cast aluminum alloy material, the precipitate calculation unit 1002 outputs the following.
Precipitate size [nm]
Precipitate density [particles/m³]
Precipitate volume fraction [volume %]

As a result, the calculation unit 630 can associate
candidate manufacturing process data sent from the extraction unit 620 with
data indicating a metallographic structure, which includes the liquidus temperature, the solid solubilities of Si, Fe, Cu, Mn, Mg, Cr, Ni, Zn, Ti, V, Pb, Sn, Bi, B, P, Zr, and Sr during solution treatment, the precipitate size, the precipitate density, and the precipitate volume fraction, and send the candidate manufacturing process data associated with the data indicating the metallographic structure to the determination unit 640.

As described above, according to the third embodiment, the same effects as those of the first embodiment can be obtained even when the design target material is the cast aluminum alloy material.

### Fourth Embodiment

In the first embodiment, an example in which the design target material is an alloy material and is specifically a wrought aluminum alloy material has been described. However, the design target material is not limited to the wrought aluminum alloy material and may be a wrought iron alloy material. Hereinafter, the fourth embodiment will be described mainly with respect to differences from the first embodiment.

### <Specific Example of Processing Performed by Analysis Unit of Manufacturing Process Search Apparatus>

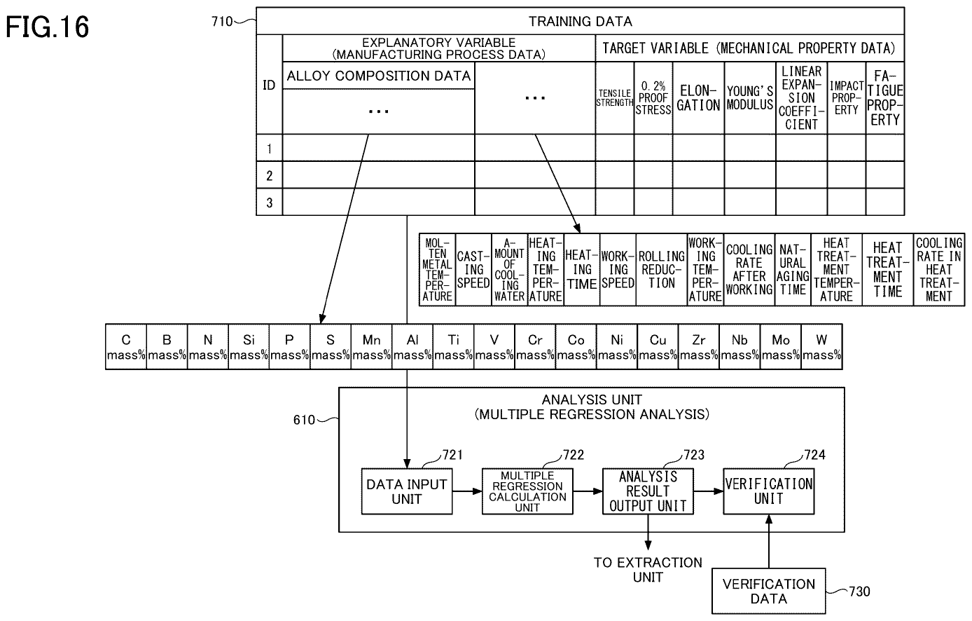

First, a specific example of processing performed by the analysis unit 610 of the manufacturing process search apparatus 400 will be described. FIG. 16 is a third diagram illustrating the specific example of the processing performed by the analysis unit.

Similarly to FIG. 7, as illustrated in FIG. 16, training data 710 read from the training data storage unit 650 by the analysis unit 610 includes, as information items, “ID”, “explanatory variable”, and “target variable”.

The “ID” stores an identification number for identifying a combination of “explanatory variable” and “target variable” included in the training data 710.

The “explanatory variable” includes, in the case of the wrought iron alloy material, “alloy composition data”, “molten metal temperature” during iron alloy pouring, “casting speed”, “amount of cooling water”, “iron alloy heating temperature” during hot working, “iron alloy heating time” during hot working, “working speed”, “rolling reduction”, “hot working temperature”, “cooling rate after hot working”, “natural aging time”, “heat treatment temperature”, “heat treatment time”, and “cooling rate in heat treatment”. The “alloy composition data” includes “C mass %”, “B mass %”, “N mass %”, “Si mass %”, “P mass %”, “Mn mass %”, “Al mass %”, “Ti mass %”, “V mass %”, “Cr masse”, “Co mass”, “Ni mass %”, “Cu mass %”, “Zr mass%”, “Nb mass %”, “Mo mass %”, and “W mass %”.

The “target variable” includes “tensile strength”, “0.2% proof stress”, “elongation”, “Young's modulus”, “linear expansion coefficient”, “impact property”, and “fatigue property”, which are mechanical properties of the wrought iron alloy material.

Note that the units included in the analysis unit 610 illustrated in FIG. 16 have already been described in the above first embodiment, and thus a description thereof will be omitted. Further, verification data 730 is the same as the training data 710, and thus a description thereof will be omitted.

### <Specific Example of Processing Performed by Calculation Unit of Manufacturing Process Search Apparatus>

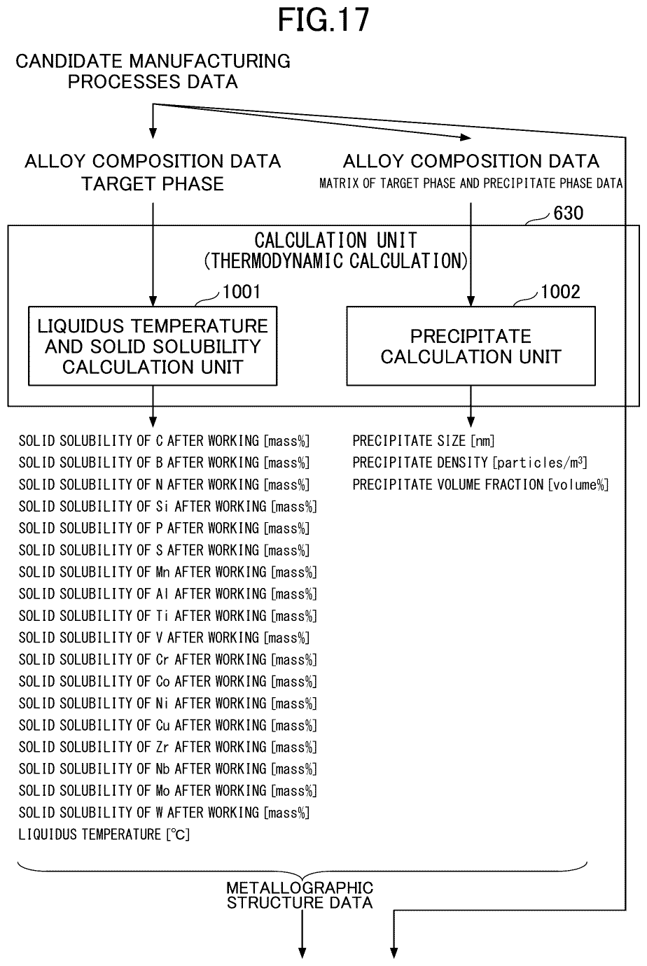

Next, a specific example of processing performed by the calculation unit 630 of the manufacturing process search apparatus 400 will be described. FIG. 17 is a third diagram illustrating the specific example of the processing performed by the calculation unit.

As illustrated in FIG. 17, in the case of the wrought iron alloy material, the liquidus temperature and solid solubility calculation unit 1001 outputs the following.
Solid solubility of C after working [mass %]
Solid solubility of B after working [mass %]
Solid solubility of N after working [mass %]
Solid solubility of Si after working [mass %]
Solid solubility of P after working [mass %]
Solid solubility of S after working [mass %]
Solid solubility of Mn after working [mass %]
Solid solubility of Al after working [mass %]
Solid solubility of Ti after working [mass %]
Solid solubility of V after working [mass %]
Solid solubility of Cr after working [mass %]
Solid solubility of Co after working [mass %]
Solid solubility of Ni after working [mass %]
Solid solubility of Cu after working [mass %]
Solid solubility of Zr after working [mass %]
Solid solubility of Nb after working [mass %]
Solid solubility of Mo after working [mass %]
Solid solubility of W after working [mass %]
Liquidus temperature [°C]

Further, in the case of the wrought iron alloy material, the precipitate calculation unit 1002 outputs the following.
Precipitate size [nm]
Precipitate density [particles/m³]
Precipitate volume fraction [volume %]

As a result, the calculation unit 630 can associate
candidate manufacturing process data sent from the extraction unit 620, with
data indicating a metallographic structure, which includes the liquidus temperature, the solid solubilities of C, B, N, Si, P, S, Mn, Al, Ti, V, Cr, Co, Ni, Cu, Zr, Nb, Mo, and W after working, the precipitate size, the precipitate density, and the precipitate volume fraction, and
send the candidate manufacturing process data associated with the data indicating the metallographic structure to the determination unit 640.

As described above, according to the fourth embodiment, the same effects as those of the first embodiment can be obtained even when the design target material is the wrought iron alloy material.

### Fifth Embodiment

In the above-described third embodiment, an example in which the design target material is an alloy material and is specifically a cast aluminum alloy material has been described. However, the design target material is not limited to the cast aluminum alloy material and may be a cast iron material. Hereinafter, the fifth embodiment will be described mainly with respect to differences from the third embodiment.

### <Specific Example of Processing Performed by Analysis Unit of Manufacturing Process Search Apparatus>

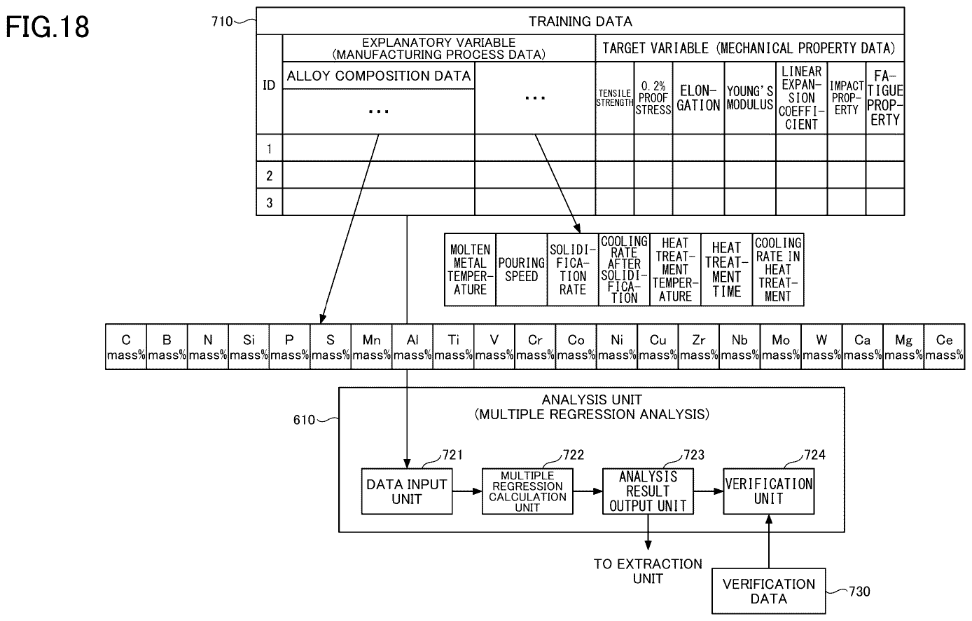

First, a specific example of processing performed by the analysis unit 610 of the manufacturing process search apparatus 400 will be described. FIG. 18 is a fourth diagram illustrating the specific example of the processing performed by the analysis unit.

Similarly to FIG. 14, as illustrated in FIG. 18, training data 710 read from the training data storage unit 650 by the analysis unit 610 includes, as information items, “ID”, “explanatory variable”, and “target variable”.

The “ID” stores an identification number for identifying a combination of “explanatory variable” and “target variable” included in the training data 710.

The “explanatory variable” includes, in the case of the cast iron material, “alloy composition data”, “molten metal temperature” during pouring, “pouring speed”, “solidification rate”, “cooling rate after solidification”, “heat treatment temperature”, “heat treatment time”, and “cooling rate in heat treatment”. The “alloy composition data” includes “C mass %”, “B mass %”, “N mass %”, “Si mass %”, “P mass %”, “S mass %”, “Mn mass %”, “Al mass %”, “Ti mass %”, “V mass %”, “Cr mass %”, “Co mass %”, “Ni mass %”, “Cu mass %”, “Zr mass %”, “Nb mass %”, “Mo mass %”, “W mass %”, “Ca mass %”, “Mg mass %”, and “Ce mass %”.

The “target variable” includes “tensile strength”, “0.2% proof stress”, “elongation”, “Young's modulus”, “linear expansion coefficient”, “impact property”, and “fatigue property”, which are mechanical properties of the cast iron material.

Note that the units included in the analysis unit 610 illustrated in FIG. 18 have already been described in the above first embodiment, and thus a description thereof will be omitted. Further, verification data 730 is the same as the training data 710, and thus a description thereof will be omitted.

### <Specific Example of Processing Performed by Calculation Unit of Manufacturing Process Search Apparatus>

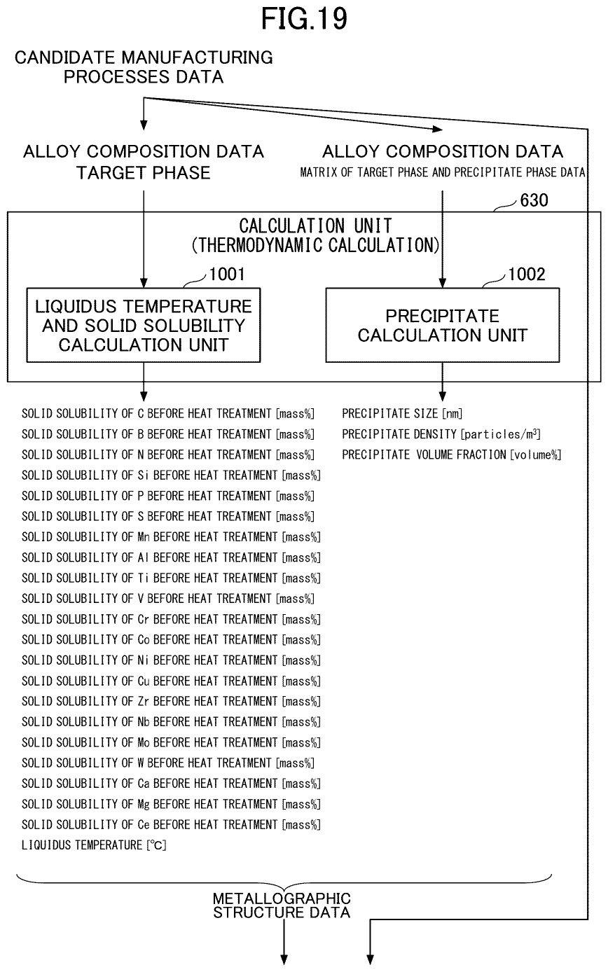

Next, a specific example of processing performed by the calculation unit 630 of the manufacturing process search apparatus 400 will be described. FIG. 19 is a fourth diagram illustrating the specific example of the processing performed by the calculation unit.

As illustrated in FIG. 19, in the case of the cast iron material, the liquidus temperature and solid solubility calculation unit 1001 outputs the following.
Solid solubility of C before heat treatment [mass %]
Solid solubility of B before heat treatment [mass %]
Solid solubility of N before heat treatment [mass %]
Solid solubility of Si before heat treatment [mass %]
Solid solubility of P before heat treatment [mass %]
Solid solubility of S before heat treatment [mass %]
Solid solubility of Mn before heat treatment [mass %]
Solid solubility of Al before heat treatment [mass %]
Solid solubility of Ti before heat treatment [mass %]
Solid solubility of V before heat treatment [mass %]
Solid solubility of Cr before heat treatment [mass %]
Solid solubility of Co before heat treatment [mass %]
Solid solubility of Ni before heat treatment [mass %]
Solid solubility of Cu before heat treatment [mass %]
Solid solubility of Zr before heat treatment [mass %]
Solid solubility of Nb before heat treatment [mass %]
Solid solubility of Mo before heat treatment [mass %]
Solid solubility of W before heat treatment [mass %]
Solid solubility of Ca before heat treatment [mass %]
Solid solubility of Mg before heat treatment [mass %]
Solid solubility of Ce before heat treatment [mass %]
Liquidus temperature [°C]

Further, in the case of a cast iron material, the precipitate calculation unit 1002 outputs the following.
Precipitate size [nm]
Precipitate density [particles/m³]
Precipitate volume fraction [volume %]

As a result, the calculation unit 630 can associate
candidate manufacturing process data sent from the extraction unit 620 with
data indicating a metallographic structure, which includes the liquidus temperature, the solid solubilities of C, B, N, Si, P, S, Mn, Al, Ti, V, Cr, Co, Ni, Cu, Zr, Nb, Mo, W, Ca, Mg, and Ce before heat treatment, the precipitate size, the precipitate density, and the precipitate volume fraction, and
send the candidate manufacturing process data associated with the data indicating the metallographic structure to the determination unit 640.

As described above, according to the fifth embodiment, the same effects as those of the above-described first embodiment can be obtained even when the design target material is the cast iron material.

### Sixth Embodiment

In the above-described first embodiment, an example in which the design target material is an alloy material and is specifically a wrought aluminum alloy material has been described. However, the design target material is not limited to the wrought aluminum alloy material and may be a wrought copper alloy material. Hereinafter, the sixth embodiment will be described mainly with respect to differences from the above-described first embodiment.

### <Specific Example of Processing Performed by Analysis Unit of Manufacturing Process Search Apparatus>

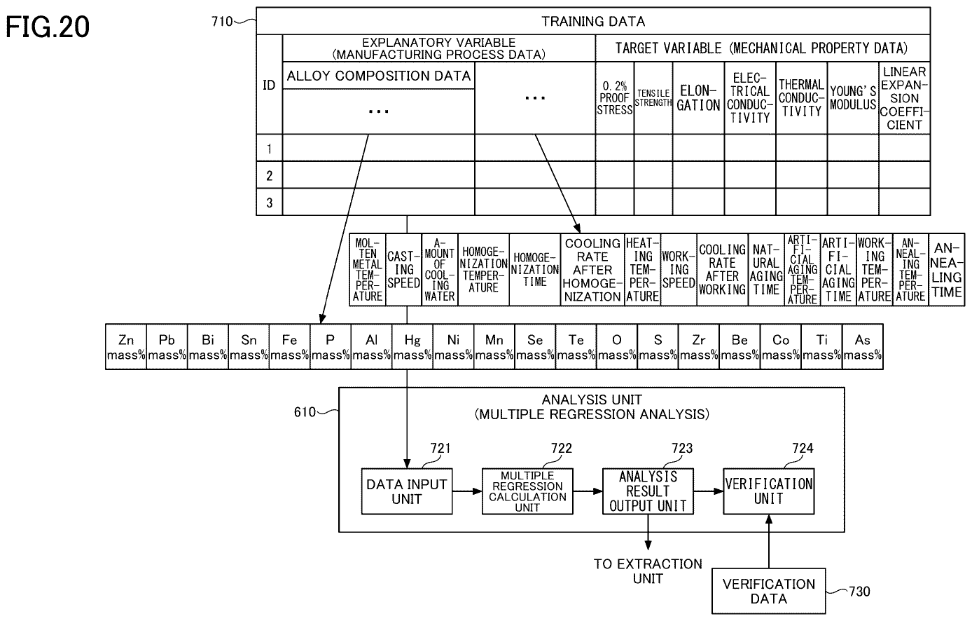

First, a specific example of processing performed by the analysis unit 610 of the manufacturing process search apparatus 400 will be described. FIG. 20 is a fifth diagram illustrating the specific example of the processing performed by the analysis unit.

Similarly to FIG. 7, as illustrated in FIG. 20, training data 710 read from the training data storage unit 650 by the analysis unit 610 includes, as information items, “ID”, “explanatory variable”, and “target variable”.

The “ID” stores an identification number for identifying a combination of “explanatory variable” and “target variable” included in the training data 710.

The “explanatory variable” includes, in the case of the wrought copper alloy material, “alloy composition data”, “molten metal temperature” during copper alloy pouring, “casting speed”, “amount of cooling water”, “homogenization temperature”, “homogenization time”, “cooling rate after homogenization”, “copper alloy heating temperature during hot working”, “working speed”, “cooling rate after working”, “natural aging time”, “artificial aging temperature”, “artificial aging time”, “hot working temperature”, “annealing temperature”, and “annealing time”. The “alloy composition data” includes “Zn mass %”, “Pb mass %”, “Bi mass %”, “Sn mass %”, “Fe mass %”, “P mass %”, “Al mass %”, “Hg mass %”, “Ni mass %”, “Mn mass %”, “Se mass %”, “Te mass %”, “O mass %”, “S mass %”, “Zr mass %”, “Be mass %”, “Co mass %”, “Ti mass %”, and “As mass %”.

The “target variable” includes “0.2% proof stress”, “tensile strength”, “elongation”, “electrical conductivity”, “thermal conductivity”, “Young's modulus”, and “linear expansion coefficient”, which are mechanical properties of the wrought copper alloy material.

Note that the units included in the analysis unit 610 illustrated in FIG. 20 have already been described in the above first embodiment, and thus a description thereof will be omitted. Further, verification data 730 is the same as the training data 710, and thus a description thereof will be omitted.

### <Specific Example of Processing Performed by Calculation Unit of Manufacturing Process Search Apparatus>

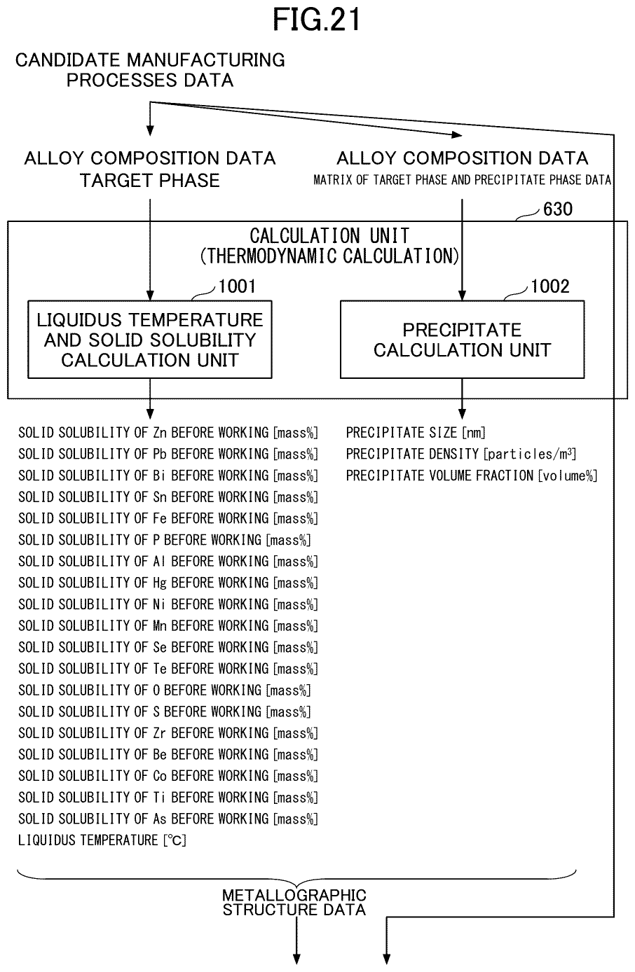

Next, a specific example of processing performed by the calculation unit 630 of the manufacturing process search apparatus 400 will be described. FIG. 21 is a fifth diagram illustrating the specific example of the processing performed by the calculation unit.

As illustrated in FIG. 21, in the case of the wrought copper alloy material, the liquidus temperature and solid solubility calculation unit 1001 outputs the following.
Solid solubility of Zn before working [mass %]
Solid solubility of Pb before working [mass %]
Solid solubility of Bi before working [mass %]
Solid solubility of Sn before working [mass %]
Solid solubility of Fe before working [mass %]
Solid solubility of P before working [mass %]
Solid solubility of Al before working [mass %]
Solid solubility of Hg before working [mass %]
Solid solubility of Ni before working [mass %]
Solid solubility of Mn before working [mass %]
Solid solubility of Se before working [mass %]
Solid solubility of Te before working [mass %]
Solid solubility of O before working [mass %]
Solid solubility of S before working [mass %]
Solid solubility of Zr before working [mass %]
Solid solubility of Be before working [mass %]
Solid solubility of Co before working [mass %]
Solid solubility of Ti before working [mass %]
Solid solubility of As before working [mass %]
Liquidus temperature [°C]

Further, in the case of the wrought copper alloy material, the precipitate calculation unit 1002 outputs the following.
Precipitate size [nm]
Precipitate density [particles/m³]
Precipitate volume fraction [volume %]

As a result, the calculation unit 630 can associate
candidate manufacturing process data sent from the extraction unit 620 with
data indicating a metallographic structure, which includes the liquidus temperature, the solid solubilities of Zn, Pb, Bi, Sn, Fe, P, Al, Hg, Ni, Mn, Se, Te, O, S, Zr, Be, Co, Ti, and As before working, the precipitate size, the precipitate density, and the precipitate volume fraction, and
send the candidate manufacturing process data associated with the data indicating the metallographic structure to the determination unit 640.

As described above, according to the sixth embodiment, the same effects as those of the above-described first embodiment can be obtained even when the design target material is the wrought copper alloy material.

### Seventh Embodiment

In the above-described third embodiment, an example in which the design target material is an alloy material and is specifically a cast aluminum alloy material has been described. However, the design target material is not limited to the cast aluminum alloy material and may be a cast copper alloy material. Hereinafter, the seventh embodiment will be described mainly with respect to differences from the above-described third embodiment.

### <Specific Example of Processing Performed by Analysis Unit of Manufacturing Process Search Apparatus>

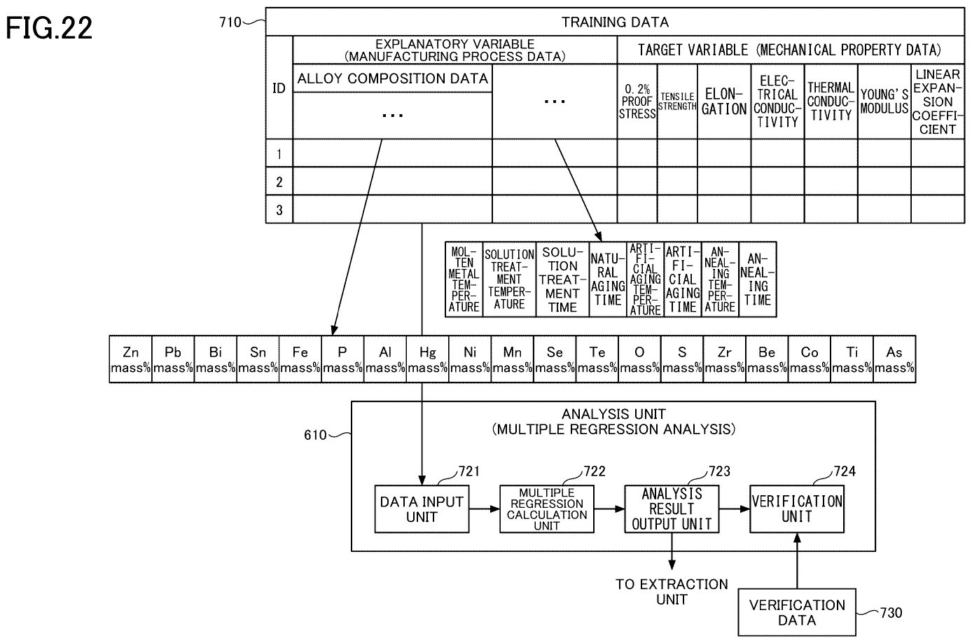

First, a specific example of processing performed by the analysis unit 610 of the manufacturing process search apparatus 400 will be described. FIG. 22 is a sixth diagram illustrating the specific example of the processing performed by the analysis unit.

Similarly to FIG. 14, as illustrated in FIG. 22, training data 710 read from the training data storage unit 650 by the analysis unit 610 includes, as information items, “ID”, “explanatory variable”, and “target variable”.

The “ID” stores an identification number for identifying a combination of “explanatory variable” and “target variable” included in the training data 710.

The “explanatory variable” includes, in the case of the cast copper alloy material, the “alloy composition data”, “molten metal temperature” during copper alloy pouring, “solution treatment temperature”, “solution treatment time”, “natural aging time”, “artificial aging temperature”, “artificial aging time”, “annealing temperature”, and “annealing time”. The “alloy composition data” includes “Zn mass %”, “Pb mass %”, “Bi mass %”, “Sn mass %”, “Fe mass %”, “P mass %”, “Al mass %”, “Hg mass %”, “Ni mass %”, “Mn mass %”, “Se mass %”, “Te mass %”, “O mass %”, “S mas s%”, “Zr mass %”, “Be mass %”, “Co mass %”, “Ti mass%”, and “As mass %”.

The “target variable” includes “0.2% proof stress”, “tensile strength”, “elongation”, “electrical conductivity”, “thermal conductivity”, “Young's modulus”, and “linear expansion coefficient”, which are mechanical properties of the cast copper alloy material.

Note that the units included in the analysis unit 610 have already been described in the above first embodiment, and thus a description thereof will be omitted.

### <Specific Example of Processing Performed by Calculation Unit of Manufacturing Process Search Apparatus>

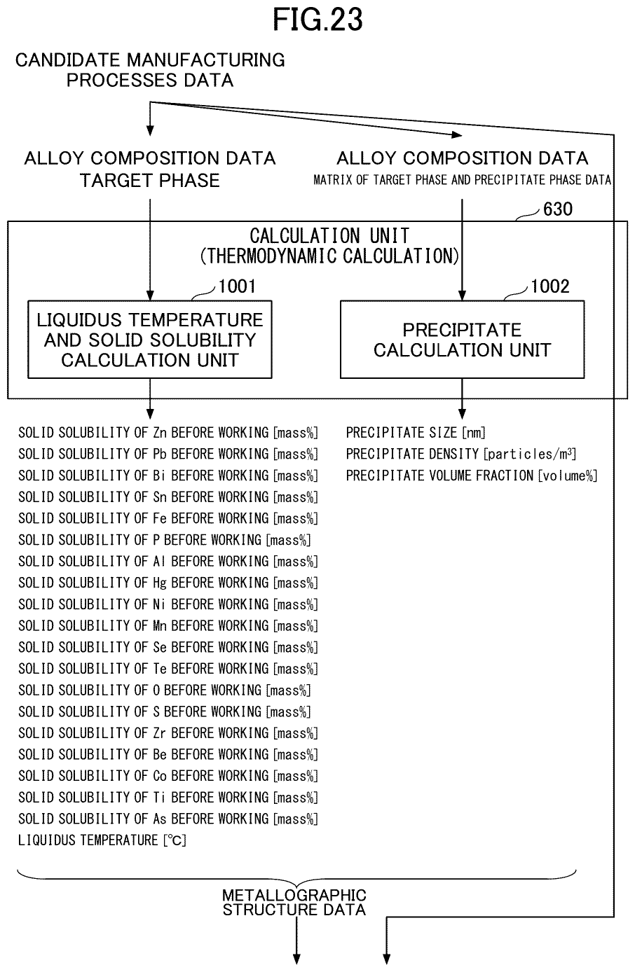

Next, a specific example of processing performed by the calculation unit 630 of the manufacturing process search apparatus 400 will be described. FIG. 23 is a sixth diagram illustrating the specific example of the processing performed by the calculation unit.

As illustrated in FIG. 23, in the case of the cast copper alloy material, the liquidus temperature and solid solubility calculation unit 1001 outputs the following.
Solid solubility of Zn before working [mass %]
Solid solubility of Pb before working [mass %]
Solid solubility of Bi before working [mass %]
Solid solubility of Sn before working [mass %]
Solid solubility of Fe before working [mass %]
Solid solubility of P before working [mass %]
Solid solubility of Al before working [mass %]
Solid solubility of Hg before working [mass %]
Solid solubility of Ni before working [mass %]
Solid solubility of Mn before working [mass %]
Solid solubility of Se before working [mass %]
Solid solubility of Te before working [mass %]
Solid solubility of O before working [mass %]
Solid solubility of S before working [mass %]
Solid solubility of Zr before working [mass %]
Solid solubility of Be before working [mass %]
Solid solubility of Co before working [mass %]
Solid solubility of Ti before working [mass %]
Solid solubility of As before working [mass %]
Liquidus temperature [°C]

Further, in the case of the cast copper alloy material, the precipitate calculation unit 1002 outputs the following.
Precipitate size [nm]
Precipitate density [particles/m³]
Precipitate volume fraction [volume %]

As a result, the calculation unit 630 can associate
candidate manufacturing process data sent from the extraction unit 620 with
data indicating a metallographic structure, which includes the liquidus temperature, the solid solubilities of Zn, Pb, Bi, Sn, Fe, P, Al, Hg, Ni, Mn, Se, Te, O, S, Zr, Be, Co, Ti, and As before working, the precipitate size, the precipitate density, and the precipitate volume fraction, and
send the candidate manufacturing process data associated with the data indicating the metallographic structure to the determination unit 640.

As described above, according to the seventh embodiment, the same effects as those of the above-described first embodiment can be obtained even when the design target material is the cast copper alloy material.

### Eighth Embodiment

In the above-described first embodiment, an example in which the design target material is an alloy material and is specifically a cast aluminum alloy material has been described. However, the design target material is not limited to the cast aluminum alloy material and may be a titanium alloy. Hereinafter, the eighth embodiment will be described mainly with respect to differences from the above-described first embodiment.

### <Specific Example of Processing Performed by Analysis Unit of Manufacturing Process Search Apparatus>

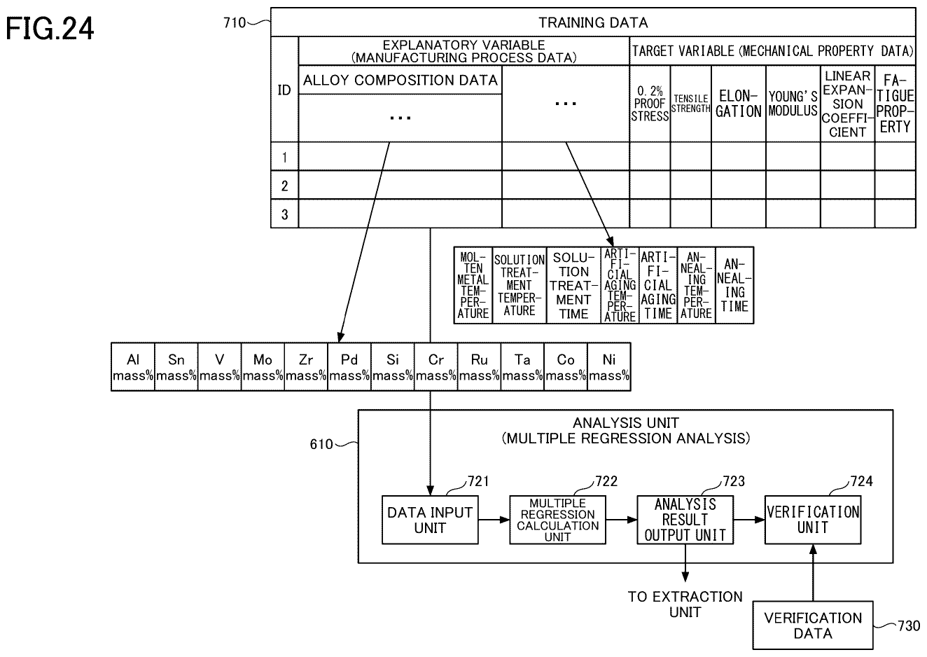

First, a specific example of processing performed by the analysis unit 610 of the manufacturing process search apparatus 400 will be described. FIG. 24 is a seventh diagram illustrating the specific example of the processing performed by the analysis unit.

Similarly to FIG. 14, as illustrated in FIG. 24, training data 710 read from the training data storage unit 650 by the analysis unit 610 includes, as information items, “ID”, “explanatory variable”, and “target variable”.

The “ID” stores an identification number for identifying a combination of “explanatory variable” and “target variable” included in the training data 710.

The “explanatory variable” includes, in the case of the titanium alloy, “alloy composition data”, “molten metal temperature”, “solution treatment temperature”, “solution treatment time”, “artificial aging temperature”, “artificial aging time”, “annealing temperature”, and “annealing time”. The “alloy composition data” includes “Al mass %”, “Sn mass %”, “V mass %”, “Mo mass %”, “Zr mass %”, “Pd mass %”, “Si mass %”, “Cr mass %”, “Ru mass %”, “Ta mass %”, “Co mass %”, and “Ni mass %.”

The “target variable” includes “0.2% proof stress”, “tensile strength”, “elongation”, “Young's modulus”, “linear expansion coefficient”, and “fatigue property”, which are mechanical properties of the titanium alloy.

Note that the units included in the analysis unit 610 illustrated in FIG. 24 have already been described in the above first embodiment, and thus a description thereof will be omitted.

Further, verification data 730 is the same as the training data 710, and thus a description thereof will be omitted.

### <Specific Example of Processing Performed by Calculation Unit of Manufacturing Process Search Apparatus>

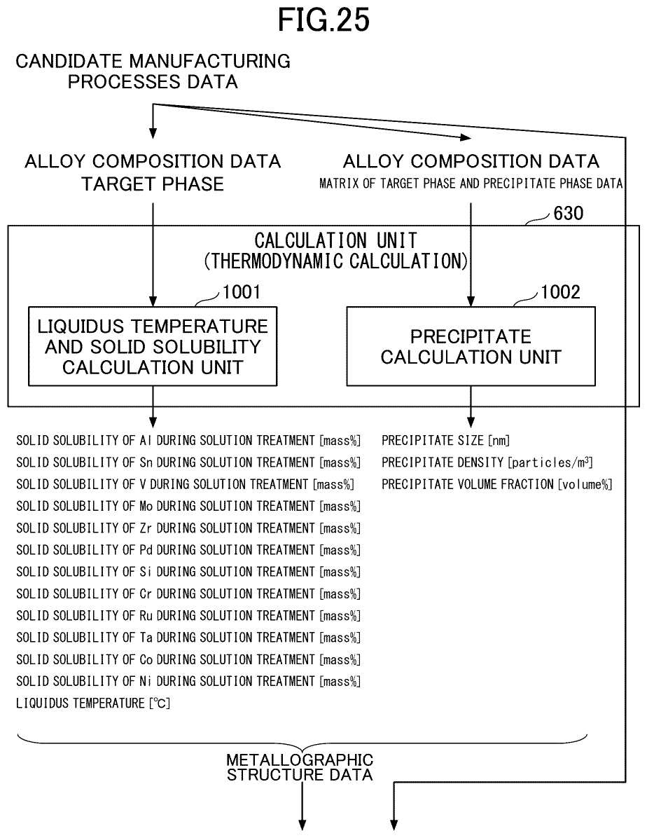

Next, a specific example of processing performed by the calculation unit 630 of the manufacturing process search apparatus 400 will be described. FIG. 25 is a seventh diagram illustrating the specific example of the processing performed by the calculation unit.

As illustrated in FIG. 25, in the case of the titanium alloy, the liquidus temperature and solid solubility calculation unit 1001 outputs the following.
Solid solubility of Al during solution treatment [mass %]
Solid solubility of Sn during solution treatment [mass %]
Solid solubility of V during solution treatment [mass %]
Solid solubility of Mo during solution treatment [mass %]
Solid solubility of Zr during solution treatment [mass %]
Solid solubility of Pd during solution treatment [mass %]
Solid solubility of Si during solution treatment [mass %]
Solid solubility of Cr during solution treatment [mass %]
Solid solubility of Ru during solution treatment [mass %]
Solid solubility of Ta during solution treatment [mass %]
Solid solubility of Co during solution treatment [mass %]
Solid solubility of Ni during solution treatment [mass %]
Liquidus temperature [°C]

Further, in the case of the titanium alloy cast material, the precipitate calculation unit 1002 outputs the following.
Precipitate size [nm]
Precipitate density [particles/m³]
Precipitate volume fraction [volume %]

As a result, the calculation unit 630 can send
candidate manufacturing process data sent from the extraction unit 620 with
data indicating a metallographic structure, which includes the liquidus temperature, the solid solubilities of Al, Sn, V, Mo, Zr, Pd, Si, Cr, Ru, Ta, Co, and Ni during solution treatment, the precipitate size, the precipitate density, the precipitate volume fraction, and
send the candidate manufacturing process data associated with the data indicating the metallographic structure to the determination unit 640.

As described above, according to the eighth embodiment, the same effects as those of the above-described first embodiment can be obtained even when the design target material is the titanium alloy.

### Other Embodiments

In each of the above-described embodiments, an example in which the relationship between manufacturing process data and mechanical property data is analyzed by multiple regression analysis has been described; however, the method of analyzing the relationship between manufacturing process data and mechanical property data is not limited thereto. For example, the relationship between manufacturing process data and mechanical property data may be analyzed by training a machine learning model using training data.

In addition, in each of the above-described embodiments, an example in which the manufacturing process search apparatus 400 is constituted by an integrated apparatus has been described; however, the manufacturing process search apparatus 400 may be constituted by a plurality of apparatuses.

The present disclosure is not limited to the configurations presented herein such as the configurations described in the embodiments, and combinations with other elements and the like are possible. These modifications can be made without departing from the scope of the present disclosure, and can be appropriately determined according to the form of application.

This application is based on and claims priority to Japanese Patent Application No. 2023-123470, filed on Jul. 28, 2023, the entire contents of which are incorporated herein by reference.

### DESCRIPTION OF THE REFERENCE NUMERALS

400: manufacturing process search apparatus
610: analysis unit
620: extraction unit
630: calculation unit
640: determination unit
710: training data
721: data input unit
722: multiple regression calculation unit
723: analysis result output unit
724: verification unit
730: verification data
921: data change unit
922: prediction unit
923: candidate extraction unit
924: candidate output unit
1001: liquidus temperature and solid solubility calculation unit
1002: precipitate calculation unit

1. A material manufacturing process search method executed by a computer for designing a design target material including a material composed of a plurality of compositions or a material manufactured by combining a plurality of manufacturing conditions, the material manufacturing process search method comprising:
analyzing a relationship between a manufacturing process of the design target material and a mechanical property of the design target material;
extracting a candidate manufacturing process for the design target material satisfying a mechanical property target value based on an analysis result obtained by analyzing the relationship;
calculating data indicating a material structure of the design target material, which is data affecting the mechanical property, by performing thermodynamic calculation for the candidate manufacturing process; and
determining appropriateness of the candidate manufacturing process based on the data indicating the material structure calculated by performing the thermodynamic calculation for the candidate manufacturing process.

2. The material manufacturing process search method according to claim 1, wherein the design target material is an inorganic material.

3. The material manufacturing process search method according to claim 2, wherein the inorganic material is an alloy material.

4. The material manufacturing process search method according to claim 3, wherein the alloy material is a wrought material or a cast material.

5. The material manufacturing process search method according to claim 4, wherein the wrought material is a wrought aluminum alloy material.

6. The material manufacturing process search method according to claim 5, wherein
the manufacturing process includes an alloy composition and a manufacturing condition,
the alloy composition includes Si, Mg, Cu, Fe, Zr, and Ti, and
the manufacturing condition includes a homogenization temperature in homogenization treatment, a pre-extrusion heating temperature in solution treatment, a thickness of an extruded cross section, an extrusion pressure, a ram speed, and a cooling water temperature.

7. The material manufacturing process search method according to claim 6, wherein the mechanical property includes tensile strength, 0.2% proof stress, and elongation.

8. The material manufacturing process search method according to claim 7, wherein
the data indicating the material structure is data indicating a metallographic structure, and
the data indicating the metallographic structure includes a liquidus temperature, a solid solubility of Mg before extrusion, a solid solubility of Si before extrusion, a precipitate size, a precipitate density, and a volume fraction.

9. The material manufacturing process search method according to claim 4, wherein the cast material is a cast aluminum alloy material.

10. The material manufacturing process search method according to claim 9, wherein
the manufacturing process includes an alloy composition and a manufacturing condition,
the alloy composition includes Si, Mg, Cu, Fe, Zr, and Ti, and
the manufacturing condition includes any of a molten metal temperature during pouring, a solution treatment temperature, a solution treatment time, a natural aging time, an artificial aging temperature, an artificial aging time, an annealing temperature, and an annealing time.

11. The material manufacturing process search method according to claim 10, wherein the mechanical property includes any of tensile strength, 0.2% proof stress, elongation, Young's modulus, a linear expansion coefficient, and a fatigue property.

12. The material manufacturing process search method according to claim 11, wherein
the data indicating the material structure is data indicating a metallographic structure, and
the data indicating the metallographic structure includes any of a liquidus temperature, solid solubilities of Si, Fe, Cu, Mn, Mg, Cr, Ni, Zn, Ti, V, Pb, Sn, Bi, B, P, Zr, and Sr during solution treatment, a precipitate size, a precipitate density, and a volume fraction.

13. The material manufacturing process search method according to claim 4, wherein the wrought material is a wrought iron alloy material.

14. The material manufacturing process search method according to claim 13, wherein
the manufacturing process includes an alloy composition and a manufacturing condition,
the alloy composition includes C, B, N, Si, P, S, Mn, Al, Ti, V, Cr, Co, Ni, Cu, Zr, Nb, Mo, and W, and
the manufacturing condition includes any of a molten metal temperature during iron alloy pouring, a casting speed, an amount of cooling water, an iron alloy heating temperature during hot working, an iron alloy heating time during hot working, a working speed, a rolling reduction, a hot working temperature, a cooling rate after hot working, a natural aging time, a heat treatment temperature, a heat treatment time, and a cooling rate in heat treatment.

15. The material manufacturing process search method according to claim 14, wherein the mechanical property includes any of tensile strength, 0.2% proof stress, elongation, Young's modulus, a linear expansion coefficient, an impact property, and a fatigue property.

16. The material manufacturing process search method according to claim 15, wherein
the data indicating the material structure is data indicating a metallographic structure, and
the data indicating the metallographic structure includes a liquidus temperature, solid solubilities of C, B, N, Si, P, S, Mn, Al, Ti, V, Cr, Co, Ni, Cu, Zr, Nb, Mo, and W after working, a precipitate size, a precipitate density, and a volume fraction.

17. The material manufacturing process search method according to claim 4, wherein the cast material is a cast iron material.

18. The material manufacturing process search method according to claim 17, wherein
the manufacturing process includes an alloy composition and a manufacturing condition,
the alloy composition includes C, B, N, Si, P, S, Mn, Al, Ti, V, Cr, Co, Ni, Cu, Zr, Nb, Mo, W, Ca, Mg, and Ce, and
the manufacturing condition includes any of a molten metal temperature during pouring, a pouring speed, a solidification rate, a cooling rate after solidification, a heat treatment temperature, a heat treatment time, and a cooling rate in heat treatment.

19. The material manufacturing process search method according to claim 18, wherein the mechanical property includes any of tensile strength, 0.2% proof stress, elongation, Young's modulus, a linear expansion coefficient, an impact property, and a fatigue property.

20. The material manufacturing process search method according to claim 19, wherein
the data indicating the material structure is data indicating a metallographic structure, and
the data indicating the metallographic structure includes a liquidus temperature, solid solubilities of C, B, N, Si, P, S, Mn, Al, Ti, V, Cr, Co, Ni, Cu, Zr, Nb, Mo, W, Ca, Mg, and Ce before heat treatment, a precipitate size, a precipitate density, and a volume fraction.

21. The material manufacturing process search method according to claim 4, wherein the wrought material is a wrought copper alloy material.

22. The material manufacturing process search method according to claim 21, wherein
the manufacturing process includes an alloy composition and a manufacturing condition,
the alloy composition includes Zn, Pb, Bi, Sn, Fe, P, Al, Hg, Ni, Mn, Se, Te, O, S, Zr, Be, Co, Ti, and As, and
the manufacturing condition includes any of a molten metal temperature during copper alloy pouring, a casting speed, an amount of cooling water, a homogenization temperature, a homogenization time, a cooling rate after homogenization, a copper alloy heating temperature during hot working, a working speed, a cooling rate after working, a natural aging time, an artificial aging temperature, an artificial aging time, a hot working temperature, an annealing temperature, and an annealing time.

23. The material manufacturing process search method according to claim 22, wherein the mechanical property includes any of 0.2% proof stress, tensile strength, elongation, electrical conductivity, thermal conductivity, Young's modulus, and a linear expansion coefficient.

24. The material manufacturing process search method according to claim 23, wherein
the data indicating the material structure is data indicating a metallographic structure, and
the data indicating the metallographic structure includes a liquidus temperature, solid solubilities of Zn, Pb, Bi, Sn, Fe, P, Al, Hg, Ni, Mn, Se, Te, O, S, Zr, Be, Co, Ti, and As before working, a precipitate size, a precipitate density, and a volume fraction.

25. The material manufacturing process search method according to claim 4, wherein the cast material is a cast copper alloy material.

26. The material manufacturing process search method according to claim 25, wherein
the manufacturing process includes an alloy composition and a manufacturing condition,
the alloy composition includes Zn, Pb, Bi, Sn, Fe, P, Al, Hg, Ni, Mn, Se, Te, O, S, Zr, Be, Co, Ti, and As, and
the manufacturing condition includes any of a molten metal temperature during pouring, a solution treatment temperature, a solution treatment time, a natural aging time, an artificial aging temperature, an artificial aging time, an annealing temperature, and an annealing time.

27. The material manufacturing process search method according to claim 26, wherein the mechanical property includes any of 0.2% proof stress, tensile strength, elongation, electrical conductivity, thermal conductivity, Young's modulus, and a linear expansion coefficient.

28. The material manufacturing process search method according to claim 27, wherein
the data indicating the material structure is data indicating a metallographic structure, and
the data indicating the metallographic structure includes any of a liquidus temperature, solid solubilities of Zn, Pb, Bi, Sn, Fe, P, Al, Hg, Ni, Mn, Se, Te, O, S, Zr, Be, Co, Ti, and As before working, a precipitate size, a precipitate density, and a volume fraction.

29. The material manufacturing process search method according to claim 3, wherein the inorganic material is a titanium alloy.

30. The material manufacturing process search method according to claim 29, wherein
the manufacturing process includes an alloy composition and a manufacturing condition,
the alloy composition includes Al, Sn, V, Mo, Zr, Pd, Si, Cr, Ru, Ta, Co, and Ni, and
the manufacturing condition includes any of a molten metal temperature during pouring, a solution treatment temperature, a solution treatment time, an artificial aging temperature, an artificial aging time, an annealing temperature, and an annealing time.

31. The material manufacturing process search method according to claim 30, wherein the mechanical property includes any of 0.2% proof stress, tensile strength, elongation, Young's modulus, a linear expansion coefficient, and a fatigue property.

32. The material manufacturing process search method according to claim 31, wherein
the data indicating the material structure is data indicating a metallographic structure, and
the data indicating the metallographic structure includes any of a liquidus temperature, solid solubilities of Al, Sn, V, Mo, Zr, Pd, Si, Cr, Ru, Ta, Co, and Ni during solution treatment, a precipitate size, a precipitate density, and a volume fraction.

33. A material manufacturing process search apparatus for designing a design target material including a material composed of a plurality of compositions or a material manufactured by combining a plurality of manufacturing conditions, the material manufacturing process search apparatus comprising:
a processor; and
a memory storing program instructions that cause the processor to:
analyze a relationship between a manufacturing process of the design target material and a mechanical property of the design target material;
extract a candidate manufacturing process for the design target material satisfying a mechanical property target value based on an analysis result obtained by analyzing the relationship;
calculate data indicating a material structure of the design target material, which is data affecting the mechanical property, by performing thermodynamic calculation for the candidate manufacturing process; and
determine appropriateness of the candidate manufacturing process based on the data indicating the material structure calculated by performing the thermodynamic calculation for the candidate manufacturing process.

34. A non-transitory computer-readable recording medium having computer-readable instructions stored thereon, which when executed, cause a computer of a material manufacturing process search apparatus for designing a design target material including a material composed of a plurality of compositions or a material manufactured by combining a plurality of manufacturing conditions to execute a process comprising:
analyzing a relationship between a manufacturing process of the design target material and a mechanical property of the design target material;
extracting a candidate manufacturing process for the design target material satisfying a mechanical property target value based on an analysis result obtained by analyzing the relationship;
calculating data indicating a material structure of the design target material, which is data affecting the mechanical property, by performing thermodynamic calculation for the candidate manufacturing process; and
determining appropriateness of the candidate manufacturing process based on the data indicating the material structure calculated by performing the thermodynamic calculation for the candidate manufacturing process.
